(19) United States
(12) Patent Application Publication
(10) Pub. No.: US 2004/0213766 A1
(43) Pub. Date: Oct. 28, 2004

(54) Compositions and methods for treating transplants

(76) Inventor: Cedric Francois, Louisville, KY (US)

Correspondence Address: EVAN LAW GROUP LLC, 566 WEST ADAMS, SUITE 350, CHICAGO, IL, 60661, US

(21) Appl. No.: 10/724,527

(22) Filed: Nov. 28, 2003

Related U.S. Application Data
(60) Provisional application No. 60/429,435, filed on Nov. 27, 2002.

(51) Int. Cl. A01N 1/00; A01N 1/02; A61K 9/127

(52) U.S. Cl. 424/93.7; 424/450; 435/1.1

(57) ABSTRACT

Methods and compositions for protecting transplants from immunorejection are provided.

### RELATED APPLICATIONS

This application claims priority to U.S. Provisional Application Ser. No. 60/429,435, filed Nov. 27, 2002, which contents are herein incorporated by reference in their entirety.

This application incorporates by reference in their entireties U.S. provisional application Ser. No. 60/380,762, filed May 14, 2002, and U.S. non-provisional application Ser. No. 10/397,048, filed Mar. 25, 2003.

### BACKGROUND

Every year 25,000 Americans receive a transplant; many after years of waiting for an available and appropriate transplant while simultaneously hoping to avoid a premature death.

Every year, 25,000 Americans who receive a transplant commence a regime of non-specific immunosuppression to prevent rejection. The cost of receiving a chance at a normal lifespan is a diminished quality of life as the recipients then suffer the negative side effects of non-specific immunosuppression. These include opportunistic infections, organ failure and post-transplant lymphoproliferative disease, thus making organ transplantation, even 5 decades after its first successful practice, a procedure afflicted with morbidity, mortality and cost.

The idea of transplantation is ancient, dating back thousands of years. Many ancient texts describe the elusive possibility of replacing missing body parts with those from a cadaver (Tilney, 1996). Not until the introduction of surgery and anesthesiology in the late 19th century, however, did this feat become closer to reality; pioneering work then established the surgical and immunological foundations of transplantation surgery. These findings eventually culminated in the first kidney transplantation in 1954 (Kann et al., 2000; Murray et al., 1956).

However, immunorejection confounded transplantation. With the introduction of cyclosporine, a clacineurin inhibitor, followed by the introduction tacrolimus, the situation was ameliorated. The possibility of transplanting tissues other than organs (e.g., hands and larynxes) once elusive, became tenable, as demonstrated by the first successful hand transplantations (Dubernard et al., 1999a; Dubernard et al., 1999b; Francois et al., 2000; Jones et al., 2000).

Transplantation Tolerance

Immunologic tolerance, the absence of a pathogenic immune response against specific foreign antigens in the absence of ongoing exogenous immunosuppression (and with the retention of immune responses against other antigens) (Adler and Turka, 2002), has been the holy grail of transplantation immunology (Helderman and Goral, 2000). The hundreds of protocols that have been developed make use of a wide variety of strategies to specifically down-regulate the immune response against foreign antigens of interest. Among the most successful are summarized in Table I.

TABLE I
Immunotherapy directed at transplantation

| | Notes/ | |
|---|---|---|
| Therapy | Applications | References |
| allogeneic | bone marrow | (Ildstad and Sachs, 1984; |
| chimerism | transplantation | Sykes, 2001; Sykes and |
| | | Sachs, 1990; Wekerle et al., |
| | | 2000; Wekerle et al., 1998) |
| T cell | with | (Kirk et al., 1999; Kirk et |
| co-stimulatory | or without donor | al., 1997; Kishimoto et al., |
| blockade | antigen | 2000; Sayegh, 1999; Sayegh |
| | | and Turka, 1998) |
| Non-mitogenic | | (Lenardo et al., 1999; |
| humanized anti-CD34 | | Thomas et al., 1997) |
| antibody (Ab) | | |
| Profound T cell | e.g., campath-1 | (Janeway et al., 2001; Kirk |
| depletioin | | et al., 2003; Salama et al., |
| | | 2001; Waldmann, 2002) |

Current strategies have in common the approach of systemic treatment of the recipient prior to, during and/or following, transplantation.

Fas-Induced Apoptosis

Apoptosis is purposeful cell death-programmed cell death, a phenomenon that is both distinct morphologically and biochemically from “unprogrammed” cell death—necrosis (Janeway et al., 2001). Apoptosis can be induced by many means, including the binding of specific ligands to their cognate receptors, as well as the imposition of certain physiological conditions

Fas and FasL are two polypeptides that interact to activate one of the best defined apoptotic pathways (Janeway et al., 2001). Fas and FasL belong to the tumor necrosis factor (TNF) family, Fas belonging to the transmembrane receptor branch and FasL to the membrane-associated cytokine branch (Nagata and Golstein, 1995). Both polypeptides contain cysteine repeats in their extracellular regions and death domains in their cytosolic tails to which adaptor polypeptides bind (Krammer et al., 1994). When a homotrimer of FasL binds Fas, Fas polypeptides trimerize, bringing together the death domains on the cytoplasmic tails (Janeway et al., 2001; Nagata and Golstein, 1995; Van Parijs and Abbas, 1998). The adaptor polypeptide, Fas-associating polypeptide with death domain (FADD), binds the activated death domains; FADD themselves bind pro-caspase 8 through a set of death effector domains (DED). When pro-caspase 8 enzymes are brought together, they transactivate and cleave themselves to release caspase 8, a protease that cleaves polypeptide chains at aspartic acid residues. Caspase 8 then cleaves and activates other caspases, eventually activating caspase 3. Caspase 3 cleaves inibitor-caspase activated DNAse (1-CAD), which enables CAD to enter the nucleus and cleave DNA (FIG. 1), a hallmark of apoptotic death (Janeway et al., 2001). Small 200 base-pair fragments of DNA result, destroying the genetic engine of the cell, leading to its ultimate demise.

Three different forms of FasL are known: membrane-bound, soluble and vesicular, each differ in their function with respect to apoptosis and immune regulation. Apoptosis is primarily mediated by the vesicular and membrane-bound forms of FasL, whereas the soluble form is ineffective in mediating apoptosis and serves as an anti-apoptotic factor by competing for the Fas receptor with membrane bound FasL (Schneider et al., 1998; Suda et al., 1993). The apoptotic activity of FasL is most efficiently mediated by its trimerization and through cell-to-cell contact, which is counterbalanced by the anti-apoptotic activity of the soluble form. The four known main roles of Fas binding to pro-apoptotic FasL are: (1) for CD4 T cells to maintain lymphocyte homeostasis, (2) for triggering the death of macrophages infected with bacteria, (3) for killing anergic B cells, and (4) for CD8 T cells to kill virally infected target cells (Janeway et al., 2001).

The critical roles that Fas-FasL interactions play in immune homeostasis, self-tolerance, and immune privilege in vital organs (Bellgrau et al., 1995; Green and Ferguson, 2001; Griffith et al., 1995) has been exploited in genetic-based immunomodulatory approach to prevent allograft rejection and induce tolerance to solid organ allografts (Ju et al., 1995; Kabelitz et al., 1993; Kang et al., 1997; Lau et al., 1996; Li et al., 1998; Swenson et al., 1998; Takeuchi et al., 1999). In some instances, alloreactive responses have been blocked, resulting in the survival of allogeneic liver, kidney, thyroid, and pancreatic islets (Arai et al., 1997; Lau et al., 1996; Matsue et al., 1999; Swenson et al., 1998; Zhang et al., 1999). While many methods to induce transplantation tolerance have been explored in the past five decades, none are yet considered to be satisfactorily safe and efficient to warrant widespread clinical use.

Fusogenic Vesicles

Small lipid vesicles (liposomes) that are fusogenic with cellular bi-lipid membranes can be used to encapsulate pharmaceutical compositions and deliver the compositions directly intracellularly. The rate of delivery can be controlled by varying the lipid vesicle composition, as well as by other means, resulting in different fusion rates. In addition, the vesicle composition can be modulated to accommodate different modes of administration. For example, small lipid vesicles can be made such that when injected into the circulation, the vesicles inefficiently fuse with endothelial cells, but can fuse efficiently to the target cells. To encourage or target fusion, other components may be added to the vesicles, such as certain polypeptides. By being loaded into a lipid vesicle, compositions are stabilized against hydrolysis.

Lipid vesicle membranes resemble plasma cell membranes; in addition, they are simple to make. Because they have an aqueous portion, lipid vesicles can encapsulate various solutions, including those containing ATP. Liposomes are made of phospholipids (amphipathic molecules) that form closed, fluid-filled spheres when mixed in water. As a liposome forms, the water-soluble molecules in the solution are encapsulated into the aqueous space in the interior of the sphere, whereas the lipid-soluble molecules in the solution are incorporated into the lipid bilayer.

Fusogenic lipid vesicles (FUVs) can adsorb to almost any cell type. Fused FUVs can exchange lipids with cell membranes. When fusion takes place, the liposomal membrane is integrated into the cell membrane and the aqueous contents of the liposome merge with the fluid in the cytosol.

Since the lipids of FUVs are incorporated into the membranes of the target cells, functional features of cell membranes may be modified at will. For example, FUVs carrying phosphatidylserine can modify the surface of T cells such that they masquerade as apoptotic cells to macrophages in culture (Borisenko et al., 2003; Fadok et al., 2001).

### SUMMARY

In a first aspect, the invention provides methods of treating a transplant, comprising administering to the transplant a T cell-apoptosis-inducing molecule, and a phospholipid which is a stable vesicle former, wherein the vesicle has a fusion rate of at least 20 vesicle fusions/second

In a second aspect, the invention provides methods of treating a transplant, comprising administering to the transplant a vesicle, which has at least in part a phospholipid which is stable vesicle former and another molecule that is either another polar lipid, PEG, a raft former or a fusion protein, and a lipid; the vesicle has a fusion rate of at least 20 vesicle fusions/second.

In a third aspect, the invention provides methods for treating a transplant by simply administering to the transplant a T cell-apoptosis-inducing molecule.

In yet another aspect, the invention provides vesicles which contain at least in part a phospholipid which is a stable vesicle former and a T cell-apoptosis-inducing molecule. The vesicle may further contain N-biotinoyl-1,2-dihexadecanoyl-sn-glycero-3-phosphoethanolamine, and a T cell-apoptosis-inducing molecule which is a chimeric polypeptide of a FasL polypeptide and at least one biotin-binding domain. In other apsects, the invention provides for transplants treated with such vesicles.

In another aspect, the invention provides vesicles consisting at least in part a T cell-apoptosis-inducing molecule,

a phospholipid which is stable vesicle former, which may be 1,2-dioleoyl-sn-glycero-3-phosphocholine, 1-palmitoyl-2-docosahexaenoyl-sn-glycero-3-phosphocholine and a mixture thereof, and

a polar lipid which is not a stable vesicle former or PEG; the polar lipid has a structure of formulas (XVII), (XVIII), (XIX), (XX), (XXI), (XXII), (XXIII), (XXV) or (XXVI):

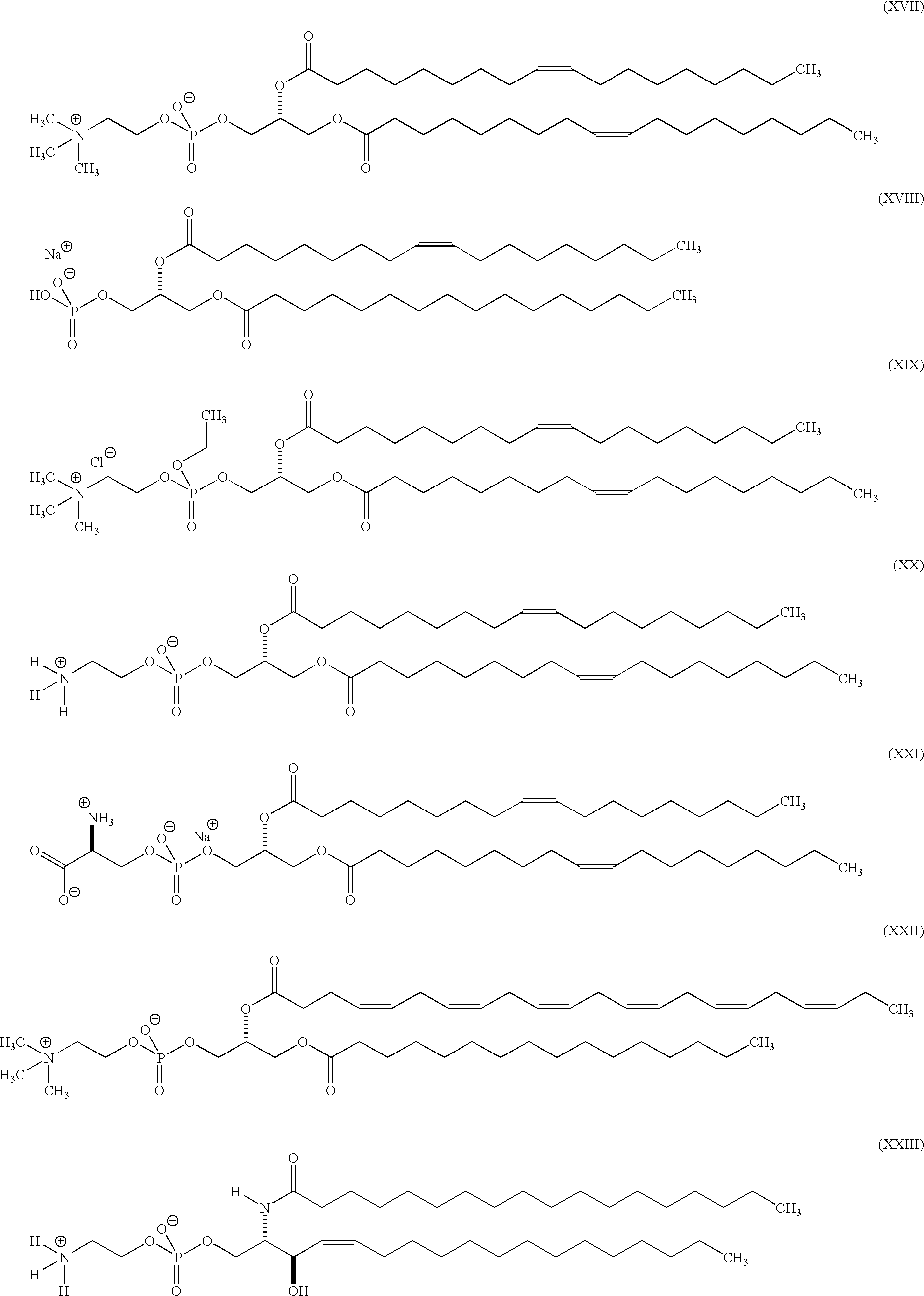

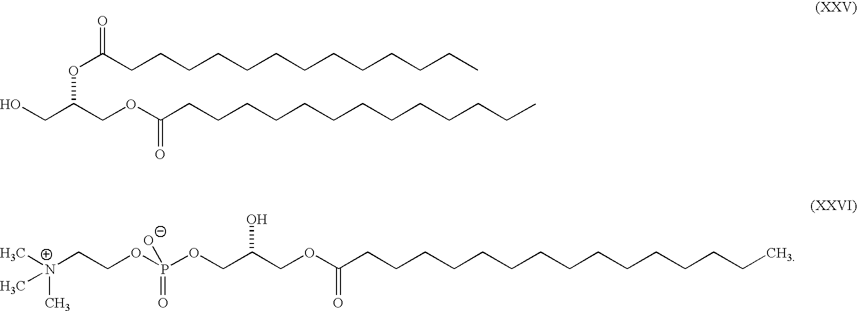

and

wherein the phospholipid which is stable vesicle former has a structure of formula (I)

X-L-Z₂ (I)

wherein X is H, or has a structure of formula (II)

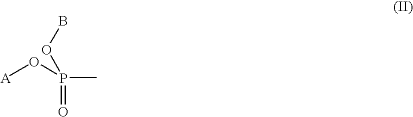

B is a cation or an alkyl group,

A is H, or has a structure selected from the group consisting of formulas (III), (IV), (V), (VI) and (VII)

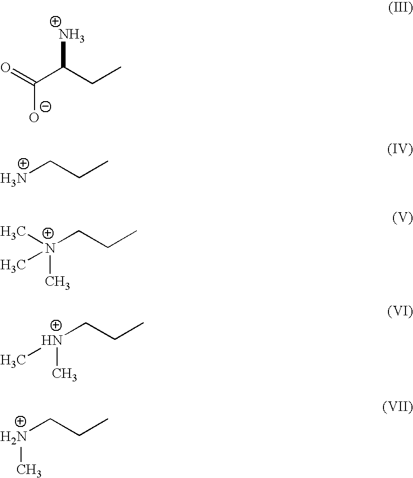

L has a structure selected from the group consisting of formulas (VIII), (IX) or (X)

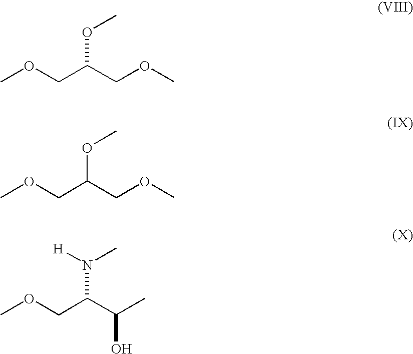

and E has a structure selected from the group consisting of (XII), (XIII), (XIV), (XV) or (XVI)

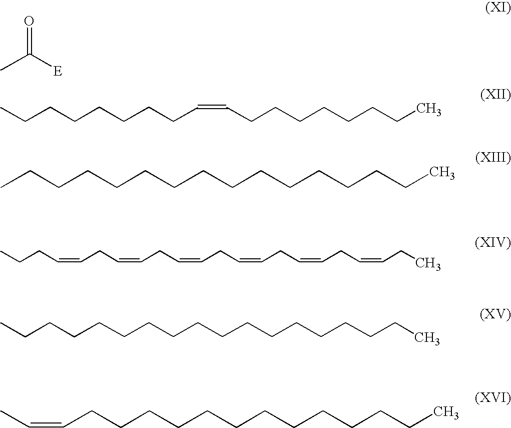

In other apsects, the invention provides for transplants treated with such vesicles.

In yet another aspect, the invention provides methods for transplanting a transplant into a recipient without administering immunosuppressive therapy.

In another aspect, the invention provides an improvement on methods of transplanting a transplant that included transplanting a transplant into a recipient, administering to the recipient immunosuppressive therapy, the improvement being contacting the transplant with a vesicle of claim 20.

In another aspect, the invention provides methods of treating a transplant, wherein the transplant is administered a T cell-apoptosis-inducing molecule, and

a vesicle, consisting of at least

a means for binding the T cell-apoptosis-inducing molecule, and

a phospholipid which is a stable vesicle former. The T cell-administration of the vesicles to the transplant. In some instances, the transplant has already been transplanted into a recipient.

In these aspects; in some embodiments, the fusion rate of the vesicles may be at least 10³ vesicle fusions/second. The T cell-apoptosis-inducing molecule may further comprise a lipid moiety, a biotin moiety, a chimeric polypeptide of avidin or streptavidin (or biotin-binding fragments thereof), or a chimeric polypeptide of FasL. The method is applicable to any transplant. In some instances, incorporation of ATP into the vesicles is desirable to maintain viability of the transplant prior to transplantation.

### DESCRIPTION OF THE FIGURES

FIG. 1 pictorially depicts the FasL-induced apoptotic pathway.

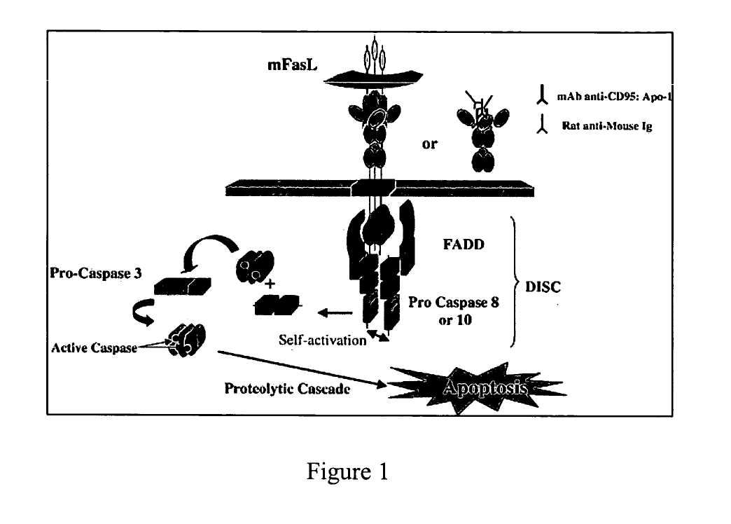

FIG. 2 shows an example of a streptavidin-apoptosis inducing chimera, SA-FasL (from Yolcu et al., 2002).

### DETAILED DESCRIPTION

The present invention solves the problem of transplant rejection by targeting activated T cells for apoptosis when they enter the transplant. “Land mines” of apoptotic molecules, such as FasL, are tethered to the endothelial membranes of the transplant, lying in wait for the unwary T cell. FasL specifically binds Fas receptors on T cells, triggering the death of the cell before the cell has the opportunity to damage the transplant.

The invention exploits the rapid and efficient display of selected exogenous polypeptides on the membranes of cells without having to genetically manipulate the host. Engineered highly fusogenic vesicles (FUVs) quickly incorporate into cell membranes, the lipids of which are modified to include specific molecules that act as tethers that bind target molecules. This unique way of tethering, for example, the extracellular domains of single-pass transmembrane polypeptides to the lipids of cell membranes, prevents the rapid internalization of the polypeptides.

Without harming or pre-treating the recipient, the endothelium of an allograft are coated with a protective veil consisting of selected exogenous molecules. In the case of T cell-apoptosis inducing polypeptides, such as FasL, cause donor-activated T cells to undergo apoptosis, circumventing allograft rejection. The invention allows for the significant reduction, if not elimination, of non-specific immunosuppression therapy after transplantation.

The advantages of this invention over other immunorejection therapies include: (1) target tissues can be quickly treated to “express” functional exogenous polypeptides in less than an hour; (2) the recipient does not require pre-treatment; (3) the materials and procedures are safe for both the treated tissue and the recipient; and (4) the polypeptides remain on the cell surface for at least two weeks, long enough to allow for the development of transplantation tolerance.

Previous attempts at using a FasL polypeptide to coat a transplant failed after 17 days. In this approach, a chimeric FasL polypeptide was engineered to contain a streptavidin domain, which bound biotin. The heart endothelial membranes were biotinylated on their integral and peripheral polypeptides by perfusing a solution of sulfo-NHS-LC-biotin. Biotinylation using LC-biotin depends on the presence of available amines on the exposed polypeptides. When a heart allograft was perfused first with sulfo-NHS-LC-biotin and then with SA-FasL, heart allograft survival was increased from 9.6+1 days to 17.4+5 days (Askenasy et al., 2003; Yolcu et al., 2002). The likely explanation of the aborted transplantation, which the present invention solves by using biotinylated lipids, is the disappearance of the FasL polypeptides from the cell surfaces. In addition, because biotinylated lipids are not attached to the cytoskeleton, they have much more lateral freedom in the external leaflet of the endothelial membranes. Since this allow the “floating” biotinylated lipids to easily approach each other, the trimerization necessary for FasL functionalization is considerably facilitated.

The present invention also makes use of the discovery that small lipid vesicles that are fusogenic with cellular bilipid membranes can deliver exogenous membrane molecules directly to cells.

Embodiments

The following embodiments exemplify the application of FasL to the methods and compositions of the invention. However, one of skill in the art will understand that other apoptosis-inducing molecules (Table II) can easily be adapted for use as desired.

TABLE II
Examples of apoptosis-inducing molecules
Apoptosis-inducing molecule

FasL
Tumor necrosis factor (TNF) receptor-1
TNF-related apoptosis inducing ligand (TRAIL)
receptor DR4
TRAIL receptor DR5

In one embodiment, the immuno-modulatory molecule FasL is coated in a dense and durable fashion onto the endothelium of the organ to be transplanted. Activated T cells express high levels of Fas on their surface. When brought in contact with FasL, an apoptotic cascade is activated, leading to certain death of the activated T cells. The methods of the invention safely and effectively down-regulate the immune response of a recipient against the foreign antigens introduced by the transplanted organ.

In another embodiment, chimeric polypeptides consisting of streptavidin (or avidin) and the extracellular domain of an apoptotic-inducing molecule, such as FasL, are engineered. The FasL domain lacks the metallopolypeptidease cleavage site, effectively preventing the production of soluble FasL. The chimeric polypeptides bind to the biotin moieties on the phospholipids via their streptaviding domains, and are consequently incorporated into the cell membranes of the endothelial cells using FUVs. Using a biotin intermediate greatly facilitates the use of transmembrane polypeptides, which are difficult to isolate and to re-incorporate into membranes, especially into those membranes that have a different lipid composition than the polypeptide's native lipid environment.

In another embodiment, chimeric polypeptides are constructed such that at least one transmembrane domain is incorporated into an apoptotic-inducing polypeptide, and then incorporated into FUVs.

In yet another embodiment, other molecules may be incorporated into the FUV membranes that allow for the specific targeting of transplants. These FUVs also contain the apoptosis-inducing molecules, but in this fashion, systemic administration of the FUVs can be accomplished without the side effect of killing substantial numbers of native T cells.

In another embodiment, FUVs are also loaded with ATP. In this way, transplant ischemia may be avoided while simultaneously incorporating the apoptosis-inducing molecules into the transplant endothelial cells. In some instances, FUVs loaded with ATP may be administered separately from the apoptosis molecule-containing FUVs.

In another embodiment, a subject is administered FUVs loaded with at least an apoptosis-inducing molecule, either systemically or directly to the transplant. Administration is repeated at regular intervals until (or if) transplant tolerance is induced.

The following, not meant to limit the invention, is presented to aid the practitioner, although other methods, techniques, cells, reagents and approaches can be used. In the first part, methods and compositions concerning FUVs containing apoptosis-inducing molecules are discussed. Next, FUVs themselves are presented. In the penultimate section, pharmaceutical compositions and administration are discussed. Finally, examples are presented that demonstrate various embodiments of the invention.

Definitions

An “apoptosis-inducing molecule” is a molecule that when contacted with a cell, induces apoptosis in that cell. The molecule may be a polypeptide, an organic molecule, a lipid, a hormone, etc., or a combination of such molecules (or moieties or fragments thereof). The molecule may be modified from its natural state; for example, a polypeptide that is an apoptosis-inducing molecule may be modified by either post-translational modifications, or using recombinant technology, to create, for example, chimeric molecules. For example, an apoptosis inducing molecule may have the parts (moieties) of: a lipid, a biotin moiety, a streptavidin or aviding moiety (or biotin-binding fragment thereof), and a FasL polypeptide, or fragment thereof, having at least the T cell-binding domains.

A “transplant” is any cell, tissue, appendage or organ that is transferred from a donor to a recipient. Examples of transplants include sperm, eggs, platelets, blood, skin, muscle, adipose tissue, nerve tissue, lymph, bone, ligament, eye, tongue, lung, trachea, heart, spleen, stomach, intestine, kidney, liver, finger, hand, toe, foot, arm and leg.

A “lipid” is a lipid that, in addition to its native structure, contains a molecular modification or addition. Such modifications include the addition of small molecules, such as biotin or FITC, polysaccharides, and heparin.

“Alkyl” (or alkyl-or alk-) refers to a substituted or unsubstituted, straight, branched or cyclic hydrocarbon chain, preferably containing of from 1 to 20 carbon atoms. Suitable examples of unsubstituted alkyl groups include methyl, ethyl, propyl, isopropyl, cyclopropyl, butyl, iso-butyl, tert-butyl, sec-butyl, cyclobutyl, pentyl, cyclopentyl, hexyl, cyclohexyl, and the like. “Alkylaryl” and “alkylheterocyclic” groups are alkyl groups covalently bonded to an aryl or heterocyclic group, respectively.

“Alkenyl” refers to a substituted or unsubstituted, straight, branched or cyclic, unsaturated hydrocarbon chain that contains at least one double bond, and preferably 2 to 20 carbon atoms. Exemplary unsubstituted alkenyl groups include ethenyl (or vinyl), 1-propenyl, 2-propenyl (or allyl) 1,3-butadienyl, hexenyl, pentenyl, 1,3,5-hexatrienyl, and the like. Preferred cycloalkenyl groups contain five to eight carbon atoms and at least one double bond. Examples of cycloalkenyl groups include cyclohexadienyl, cyclohexenyl, cyclopentenyl, cycloheptenyl, cyclooctenyl, cyclohexadienyl, cycloheptadienyl, cyclooctatrienyl and the like.

“Alkoxy” refers to a substituted or unsubstituted, —O— alkyl group. Exemplary alkoxy groups include methoxy, ethoxy, n-propoxy, isopropoxy, n-butoxy, t-butoxy, and the like.

“Aryl” refers to any monovalent aromatic carbocyclic or heteroaromatic group, preferably of 3 to 10 carbon atoms. The aryl group can be bicyclic (i.e. phenyl (or Ph)) or polycyclic (i.e. naphthyl) and can be unsubstituted or substituted. Preferred aryl groups include phenyl, naphthyl, furyl, thienyl, pyridyl, indolyl, quinolinyl or isoquinolinyl.

“Amino” refers to an unsubstituted or substituted-NRR′ group. The amine can be primary (—NH2), secondary (—NHR) or tertiary (—NRR′), depending on the number of substituents (R or R′). Examples of substituted amino groups include methylamino, dimethylamino, ethylamino, diethylamino, 2-propylamino, 1-propylamino, di (n-propyl) amino, di (iso-propyl) amino, methyl-n-propylamino, t-butylamino, anilino, and the like.

“Heterocyclic radical” refers to a stable, saturated, partially unsaturated, or aromatic ring, preferably containing 5 to 10, more preferably 5 or 6, atoms. The ring can be substituted 1 or more times (preferably 1, 2, 3, 4 or 5 times) with a substituent. The ring can be mono-, bi-or polycyclic. The heterocyclic group consists of carbon atoms and from 1 to 3 heteroatoms independently selected from the group consisting of nitrogen, oxygen, and sulfur. The heteroatoms can be protected or unprotected. Examples of useful heterocyclic groups include substituted or unsubstituted, protected or unprotected acridine, benzathiazoline, benzimidazole, benzofuran, benzothiophene, benzthiazole, benzothiophenyl, carbazole, cinnoline, furan, imidazole, 1H-indazole, indole, isoindole, isoquinoline, isothiazole, morpholine, oxazole (i.e. 1,2,3-oxadiazole), phenazine, phenothiazine, phenoxazine, phthalazine, piperazine, pteridine, purine, pyrazine, pyrazole, pyridazine, pyridine, pyrimidine, pyrrole, quinazoline, quinoline, quinoxaline, thiazole, 1,3,4-thiadiazole, thiophene, 1,3,5-triazines, triazole (i.e. 1,2,3-triazole), and the like.

“substituted” means that the moiety contains at least one, preferably 1-3 substituent (s). Suitable substituents include hydrogen (H) and hydroxyl (—OH), amino (—NH2), oxy (-0-), carbonyl (—CO—), thiol, alkyl, alkenyl, alkynyl, alkoxy, halo, nitrile, nitro, aryl and heterocyclic groups. These substituents can optionally be further substituted with 1-3 substituents. Examples of substituted substituents carboxylate, alkoxycarbonyl, alkylaryl, aralkyl, alkylheterocyclic, and the like.

Apoptosis-Inducing Molecule Engineering

A facile method of introducing exogenous polypeptides in endogenous cell membranes exploits the strong, non-covalent interaction between streptavidin and biotin. This method avoids problems that may occur when trying to incorporate purified polypeptides into lipid membranes.

For example, streptavidin is incorporated into a pre-selected apoptosis-inducing polypeptide, or apoptosis-active fragment (death fragment) thereof, such that the death fragment is available to the target cells, such as T cells. In most cases, streptavidin is readily incorporated into either the amino- or carboxy-terminus of the polypeptide, although in some (albeit rare) instances, incorporation into the interior of the polypeptide may be optimal. The death-inducing activity can be assayed in vitro before use to confirm that apoptotic activity is not disrupted (see In vitro death assays, below). One example of an appropriate streptavidin sequence is given in Tables III (polynucleotide; SEQ ID NO:1) and IV SEQ ID NO:2, polypeptide).

TABLE III

Polynucleotide sequence of an artificial gene for streptavidin
(SEQ ID NO: 1) (Thompson and Weber, 1993) (GenBank Accession
X65082)

| | |
|---|---|
| gaattcatat ggctgaagct ggtatcaccg gtacttggta caaccagctg gggtctacct | 60 |
| tcatcgttac cgctggtgct gacggtgcac tgaccggtac ttacgaaagc gctgttggta | 120 |
| acgctgaaag ccgttatgtt ctgaccggtc gttacgactc tgctccggct accgacggtt | 180 |
| ctggtactgc tctgggttgg accgttgctt ggaaaaacaa ctaccgtaac gctcactctg | 240 |
| ctaccacctg gtctggccag tacgttggtg gtgctgaagc tcgtatcaac acccagtggc | 300 |
| tgctgacctc tggtaccacc gaagctaacg cttggaaatc taccctggtt ggtcacgaca | 360 |
| cgttcaccaa agttaaaccg tctgctgctt ctatctaga | 399 |

TABLE IV

Polypeptide sequence of streptavidin as encoded by SEQ ID
NO: 1 (SEQ ID NO: 2) (Thompson and Weber, 1993) (GenBank
Accession CAA46210)

```
Met Ala Glu Ala Gly Ile Thr Gly Thr Trp Tyr Asn Gln Leu Gly Ser
1 5 10 15
Thr Phe Ile Val Thr Ala Gly Ala Asp Gly Ala Leu Thr Gly Thr Tyr
                        20 25 30
Glu Ser Ala Val Gly Asn Ala Glu Ser Arg Tyr Val Leu Thr Gly Arg
                35 40 45
Tyr Asp Ser Ala Pro Ala Thr Asp Gly Ser Gly Thr Ala Leu Gly Trp
        50 55 60
Thr Val Ala Trp Lys Asn Asn Tyr Arg Asn Ala His Ser Ala Thr Thr
65 70 75 80
Trp Ser Gly Gln Tyr Val Gly Gly Ala Glu Ala Arg Ile Asn Thr Gln
                                85 90 95
Trp Leu Leu Thr Ser Gly Thr Thr Glu Ala Asn Ala Trp Lys Ser Thr
                        100 105 110
Leu Val Gly His Asp Thr Phe Thr Lys Val Lys Pro Ser Ala Ala Ser
                115 120 125
```

Although binding biotin with less affinity, avidin may also be used. In some instances avidin can be advantageous in those situations in which streptavidin significantly interferes with the death-inducing activity of the desired apoptosis-inducing polypeptide. The polynucleotide and polypeptide sequences of avidin are given in Tables VII (SEQ ID NO:5) and VIII (SEQ ID NO:6), respectively.

TABLE V

Polynucleotide (cDNA) sequence of chicken avidin (SEQ ID NO: 5)
(GenBank Accession X05343)

| | |
|---|---|
| gaattccgca aggagcacac ccggctgtcc acctgctgca gagatggtgc acgcaacctc | 60 |
| cccgctgctg ctgctgctgc tgctcagcct ggctctggtg gctcccggcc tctctgccag | 120 |
| aaagtgctcg ctgactggga aatggaccaa cgatctgggc tccaacatga ccatcggggc | 180 |
| tgtgaacagc agaggtgaat tcacaggcac ctacatcaca gccgtaacag ccacatcaaa | 240 |
| tgagatcaaa gagtcaccac tgcatgggac acaaaacacc atcaacaaga ggacccagcc | 300 |
| cacctttggc ttcaccgtca attggaagtt ttcagagtcc accactgtct tcacgggcca | 360 |
| gtgcttcata gacaggaatg ggaaggaggt cctgaagacc atgtggctgc tgcggtcaag | 420 |
| tgttaatgac attggtgatg actggaaagc taccagggtc ggcatcaaca tcttcactcg | 480 |
| cctgcgcaca cagaaggagt gaggatggcc ccgcaaagcc agcaacaatg ccggagtgct | 540 |
| gacactgctt gtgatattcc tcccaataaa gctttgcctc agacaaaaaa aaaaaaagga | 600 |
| attc | 604 |

TABLE VI

Polypeptide sequence of chicken avidin (SEQ ID NO: 6)
(GenBank Accession CAA28954)

```
Met Val His Ala Thr Ser Pro Leu Leu Leu Leu Leu Leu Leu Ser Leu
1 5 10 15
Ala Leu Val Ala Pro Gly Leu Ser Ala Arg Lys Cys Ser Leu Thr Gly
                        20 25 30
Lys Trp Thr Asn Asp Leu Gly Ser Asn Met Thr Ile Gly Ala Val Asn
                35 40 45
Ser Arg Gly Glu Phe Thr Gly Thr Tyr Ile Thr Ala Val Thr Ala Thr
        50 55 60
Ser Asn Glu Ile Lys Glu Ser Pro Leu His Gly Thr Gln Asn Thr Ile
65 70 75 80
Asn Lys Arg Thr Gln Pro Thr Phe Gly Phe Thr Val Asn Trp Lys Phe
                                85 90 95
Ser Glu Ser Thr Thr Val Phe Thr Gly Gln Cys Phe Ile Asp Arg Asn
                        100 105 110
Gly Lys Glu Val Leu Lys Thr Met Trp Leu Leu Arg Ser Ser Val Asn
                115 120 125
Asp Ile Gly Asp Asp Trp Lys Ala Thr Arg Val Gly Ile Asn Ile Phe
        130 135 140
Thr Arg Leu Arg Thr Gln Lys Glu
145 150
```

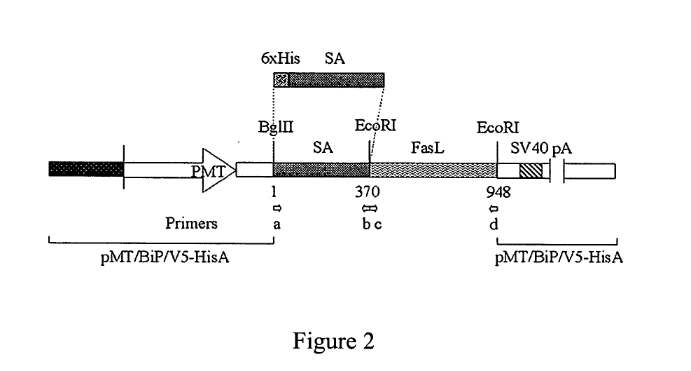

For example, chimeric streptavidin containing FasL polypeptides (SA-FasL) have been made; other polypeptides can also be made in the same manner. In one version of SA-FasL, the chimera lacks the metalloproteinase binding site of wild-type FasL (Askenasy et al., 2003; Yolcu et al., 2002). One method to engineer this molecule, a chimeric gene consisting of the biotin binding and tetramer-forming domains of streptavidin and extracellular portions of rat FasL, is to use gene-specific primers in PCR (Yolcu et al., 2002; FIG. 2). Genomic DNA is isolated from Streptomyces avidinii (American Type Culture Collection; Manassus, Va.), and 0.2 μg of this DNA is used as template for amplification using primers specific for core streptavidin in polymerase chain reactions (PCRs) (Pahler et al., 1987). The 5 [symbol] primer includes sequences for BglII and six histidine residues to allow cloning in frame with the Drosophila secretion signal (BiP) for expression as a secreted protein and purification using Ni-affinity columns. The extracellular domain of rat FasL without the metalloproteinase site is cloned using a FasL cDNA clone isolated from ConA-activated splenocytes as a template and a sense primer to the 5 [symbol] end of the extracellular region, FasL6 (nucleotides 428-453) containing an EcoRI site and an anti-sense primer to the 3 [symbol] end untranslated region of FasL, FasL2 (nucleotides 977-998) containing an EcoRI site in frame with streptavidin, in PCR. The chimeric gene is subcloned into pMT/BiP/V5-His CuSO₄-inducible vector for expression in the Drosophila Expression System (DES®) from Invitrogen (Calsbad, Calif.). The sequence of rat and human FasL is given in Tables V (polynucleotide, SEQ ID NOs:5 and 7) and VI (polypeptide, SEQ ID NOs:6 and 8).

TABLE VII

Polynucleotide sequence of rat FasL (SEQ ID NO: 5) and human
FasL (SEQ ID NO: 7) (GenBank Accessions NM_012908 and U11821,
respecively)

| | |
|---|---|
| (Rat) | |
| gaattcatat ggctgaagct ggtatcaccg gtacttggta caaccagctg gggtctacct | 60 |
| tcatcgttac cgctggtgct gacggtgcac tgaccggtac ttacgaaagc gctgttggta | 120 |
| acgctgaaag ccgttatgtt ctgaccggtc gttacgactc tgctccggct accgacggtt | 180 |
| ctggtactgc tctgggttgg accgttgctt ggaaaaacaa ctaccgtaac gctcactctg | 240 |
| ctaccacctg gtctggccag tacgttggtg gtgctgaagc tcgtatcaac acccagtggc | 300 |
| tgctgacctc tggtaccacc gaagctaacg cttggaaatc taccctggtt ggtcacgaca | 360 |
| cgttcaccaa agttaaaccg tctgctgctt ctatctaga | 399 |
| (Human) | |
| tctagactca ggactgagaa gaagtaaaac cgtttgctgg ggctggcctg actcaccagc | 60 |
| tgccatgcag cagcccttca attacccata tccccagatc tactgggtgg acagcagtgc | 120 |
| cagctctccc tgggcccctc caggcacagt tcttccctgt ccaacctctg tgcccagaag | 180 |
| gcctggtcaa aggaggccac caccaccacc gccaccgcca ccactaccac ctccgccgcc | 240 |
| gccgccacca ctgcctccac taccgctgcc acccctgaag aagagaggga accacagcac | 300 |
| aggcctgtgt ctccttgtga tgtttttcat ggttctggtt gccttggtag gattgggcct | 360 |
| ggggatgttt cagctcttcc acctacagaa ggagctggca gaactccgag agtctaccag | 420 |
| ccagatgcac acagcatcat ctttggagaa gcaaataggc caccccagtc caccccctga | 480 |
| aaaaaaggag ctgaggaaag tggcccattt aacaggcaag tccaactcaa ggtccatgcc | 540 |
| tctggaatgg gaagacacct atggaattgt cctgctttct ggagtgaagt ataagaaggg | 600 |
| tggccttgtg atcaatgaaa ctgggctgta ctttgtatat tccaaagtat acttccgggg | 660 |
| tcaatcttgc aacaacctgc ccctgagcca caaggtctac atgaggaact ctaagtatcc | 720 |
| ccaggatctg gtgatgatgg aggggaagat gatgagctac tgcactactg ggcagatgtg | 780 |
| ggcccgcagc agctacctgg gggcagtgtt caatcttacc agtgctgatc atttatatgt | 840 |
| caacgtatct gagctctctc tggtcaattt tgaggaatct cagacgtttt tcggcttata | 900 |
| taagctctaa gagaagcact ttgggattct ttccattatg attctttgtt acaggcaccg | 960 |
| agatgttcta ga | 972 |

TABLE VIII

Polypeptide sequence of rat FasL (SEQ ID NO: 6) and human
FasL (SEQ ID NO: 8) (GenBank Accessions NP_037040 and
AAC50124, respectively)

```
(Rat)
Met Gln Gln Pro Val Asn Tyr Pro Cys Pro Gln Ile Tyr Trp Val Asp
1 5 10 15
Ser Ser Ala Thr Ser Pro Trp Ala Pro Pro Gly Ser Val Phe Ser Cys
                        20 25 30
Pro Ser Ser Gly Pro Arg Gly Pro Gly Gln Arg Arg Pro Pro Pro Pro
                35 40 45
Pro Pro Pro Pro Ser Pro Leu Pro Pro Pro Ser Gln Pro Pro Pro Leu
        50 55 60
Pro Pro Leu Ser Pro Leu Lys Lys Lys Asp Asn Ile Glu Leu Trp Leu
65 70 75 80
Pro Val Ile Phe Phe Met Val Leu Val Ala Leu Val Gly Met Gly Leu
                                85 90 95
Gly Met Tyr Gln Leu Phe His Leu Gln Lys Glu Leu Ala Glu Leu Arg
                        100 105 110
Glu Phe Thr Asn His Ser Leu Arg Val Ser Ser Phe Glu Lys Gln Ile
                115 120 125
Ala Asn Pro Ser Thr Pro Ser Glu Thr Lys Lys Pro Arg Ser Val Ala
        130 135 140
His Leu Thr Gly Asn Pro Arg Ser Arg Ser Ile Pro Leu Glu Trp Glu
145 150 155 160
Asp Thr Tyr Gly Thr Ala Leu Ile Ser Gly Val Lys Tyr Lys Lys Gly
                                165 170 175
Gly Leu Val Ile Asn Glu Ala Gly Leu Tyr Phe Val Tyr Ser Lys Val
                        180 185 190
Tyr Phe Arg Gly Gln Ser Cys Asn Ser Gln Pro Leu Ser His Lys Val
                195 200 205
Tyr Met Arg Asn Phe Lys Tyr Pro Gly Asp Leu Val Leu Met Glu Glu
        210 215 220
Lys Lys Leu Asn Tyr Cys Thr Thr Gly Gln Ile Trp Ala His Ser Ser
225 230 235 240
Tyr Leu Gly Ala Val Phe Asn Leu Thr Val Ala Asp His Leu Tyr Val
                                245 250 255
Asn Ile Ser Gln Leu Ser Leu Ile Asn Phe Glu Glu Ser Lys Thr Phe
                        260 265 270
Phe Gly Leu Tyr Lys Leu
                275
(Human)
Met Gln Gln Pro Phe Asn Tyr Pro Tyr Pro Gln Ile Tyr Trp Val Asp
1 5 10 15
Ser Ser Ala Ser Ser Pro Trp Ala Pro Pro Gly Thr Val Leu Pro Cys
                        20 25 30
Pro Thr Ser Val Pro Arg Arg Pro Gly Gln Arg Arg Pro Pro Pro Pro
                35 40 45
Pro Pro Pro Pro Pro Leu Pro Pro Pro Pro Pro Pro Pro Pro Leu Pro
        50 55 60
Pro Leu Pro Leu Pro Pro Leu Lys Lys Arg Gly Asn His Ser Thr Gly
65 70 75 80
Leu Cys Leu Leu Val Met Phe Phe Met Val Leu Val Ala Leu Val Gly
                                85 90 95
Leu Gly Leu Gly Met Phe Gln Leu Phe His Leu Gln Lys Glu Leu Ala
                        100 105 110
Glu Leu Arg Glu Ser Thr Ser Gln Met His Thr Ala Ser Ser Leu Glu
                115 120 125
Lys Gln Ile Gly His Pro Ser Pro Pro Pro Glu Lys Lys Glu Leu Arg
        130 135 140
Lys Val Ala His Leu Thr Gly Lys Ser Asn Ser Arg Ser Met Pro Leu
145 150 155 160
Glu Trp Glu Asp Thr Tyr Gly Ile Val Leu Leu Ser Gly Val Lys Tyr
                                165 170 175
Lys Lys Gly Gly Leu Val Ile Asn Glu Thr Gly Leu Tyr Phe Val Tyr
                        180 185 190
Ser Lys Val Tyr Phe Arg Gly Gln Ser Cys Asn Asn Leu Pro Leu Ser
                195 200 205
His Lys Val Tyr Met Arg Asn Ser Lys Tyr Pro Gln Asp Leu Val Met
        210 215 220
Met Glu Gly Lys Met Met Ser Tyr Cys Thr Thr Gly Gln Met Trp Ala
225 230 235 240
Arg Ser Ser Tyr Leu Gly Ala Val Phe Asn Leu Thr Ser Ala Asp His
                                245 250 255
Leu Tyr Val Asn Val Ser Glu Leu Ser Leu Val Asn Phe Glu Glu Ser
                        260 265 270
Gln Thr Phe Phe Gly Leu Tyr Lys Leu
                275 280
```

Other “lock and key” molecular interactions can also be exploited in an analogous fashion. Preferably, one of these two molecules is easily conjugated to a lipid, or easily incorporated into FUV membranes. For example, fluorescein (FITC)-tagged lipids are commercially available (e.g., from Molecular Probes; Eugene, Oreg.). In this case, the FITC-tagged lipids are incorporated into FUVs, and the apoptosis-inducing polypeptide, such as FasL, is engineered to contain a Fab fragment of an anti-FITC antibody. Such an approach can be advantageous when multimerization of the apoptosis-inducing molecules are necessary; in this case, one polypeptide can contain the light chain, while the other, the heavy chain.

In Vitro Death Assays

To ascertain that a modified apoptosis-inducing molecule, or to determine if a candidate molecule does induce apoptosis, the following assays, or modifications thereof, can be performed.

In Vitro Assay

In this assay, endothelial cells are first contacted with FUVs containing either the tether of choice (e.g., biotin) or the apoptosis-inducing molecule candidate itself. In the case of tethers, the apoptosis-inducing candidate molecule, such as SA-FasL, is then attached to the cell membranes. The ability of the coated endothelial cells to trigger apoptosis is then assayed by exposing the cells to activated T cells. The experimental design is summarized in Table IX.

TABLE IX
Experimental design

| | Experimental | | |
|---|---|---|---|
| | conditions | | |
| | Vesicle | SA- | |
| Group | biotinylation | FasL | Experimental role |
| 1 | No | Yes | Non-specific binding control for |
| | | | the candidate apoptosis-inducing molecule |
| | | | binding to endothelial cells. |
| 2 | No | Yes | Non-specific binding control for |
| | | | candidate apoptosis-inducing molecule |
| | | | binding to endothelial cells that have |
| | | | been exposed to non-biotinylated FUVs |
| 3 | Yes | Yes | Experimental group to demonstrate |
| | | | candidate apoptosis- |
| | | | inducing molecule activity |

The assay is here exemplified by biotin tethers and SA-FasL.

Preparation of Groups

HUVECs (1×10⁵/well) are prepared and incubated for 1 hour with Hank's 15 balanced salt solution (HBSS) or biotinylated FUVs, as indicated in Table IX. Following incubation, the cells are washed 3 times with HBSS and then incubated with 100 ng in 1 ml of SA-FasL (MW 32 kDa) for 15 minutes at 37° C.

After washing 3 more times with HBSS, activated T cells, isolated and activated according to established protocols (e.g., see methods in (Levine et al., 2002; von Andrian, 2002)) are added to the coated endothelial cells. The cultures are then observed over time for apoptosis of the T cells, which can be measured morphologically or biochemically (Janeway et al., 2001). If apoptosis is observed only in the experimental cells, then the candidate apoptosis-inducing molecule is inducing apoptosis. One advantage of this assay is the ability to monitor the effects of FUV incorporation into the endothelial cells; such effects include, e.g., necrosis.

Assay Based on Allograft Immunorejection

Activated T cells that contact SA-FasL-functionalized lymphocytes (splenocytes—not endothelia) undergo apoptosis in a classic mixed lymphocyte reaction (MLR) assay (Shirwan et al., 1997; Yolcu et al., 2002). This assay can be adapted to test candidate apoptosis-inducing molecules.

This assay exploits transplant immunorejection between two strains of rats, ACI and Wistar Furth. In three experimental groups, splenocytes from ACI-sensitized WF rats are harvested and then exposed to endothelial ACI cells in vitro. Next, the WF splenocytes are separated and adoptively transferred to a nude recipient rat (PVG rnu/rnu). The nude rat then receives a transplant of an ACI-vascularized skin flap; rejection of the skin flap is then be monitored. The experimental design is summarized in Table IX

Group 1 (Rat Preparation and Surgical Procedures will be the Same for all Groups)

On Day −7, ACI rats are injected with 107 splenocytes harvested from ACI rats. Free epigastric groin flaps from ACI rat donors will be raised as previously described (Fernandez-Botran et al., 2002). Preferably, the flaps measure 5 cm² and consist of skin, panniculus carnosus muscle, subcutaneous fat, epigastric fat pad, inguinal lymph nodes and femoral vessels. The animals are anesthetized with sodium pentobarbital (60 mg/kg, administered intraperitoneally) and shaved in the groin area. Access to the flap is by standard groin incision. The femoral artery and vein is dissected from the inguinal ligament to the bifurcation of the inferior epigastric vessels. The distal ends of the femoral vessels are ligated using, e.g., 8-0-nylon. The isolated flap is then flushed with heparinized lactated Ringer's solution through the femoral artery for 10 minutes; venous return is not interrupted. At the end of flushing, the proximal ends of femoral vessels are clamped, and the flap raised in its entirety with the epigastric vessels and fat pad.

TABLE IX
Experimental design

| | Endothelial-Lymphocyte | | |
|---|---|---|---|
| | Reaction | | |
| | Stimulators | Responders | |
| Group | (endothelial) | (splenocytes) | Experimental role |
| 1 | ACI | ACI | Negative control, demonstrating |
| | | | lack of graft rejection |
| 2 | ACI | WF | Positive control, demonstrating |
| | | | graft rejection |
| 3 | ACI + SA- | WF | Experimental, demonstrating |
| | FasL | | protective effect of the candidate |
| | | | apoptosis-inducing molecule. |
| | | | If diminished graft rejection is |
| | | | observed, then the candidate |
| | | | molecule is likely to be |
| | | | responsible for inducing apoptosis. |

On Day −4, nude rats are transplanted with ACI skin flaps in the neck area. Nude rat recipients are anesthetized with isoflourane 2-5% and shaved in the ventral aspect of the neck region. The right external carotid artery (ECA) and external jugular vein (EJV) are exposed, and the ECA carefully separated from the cervical sympathetic plexus, clamped proximally, and cut. The EJV is clamped distally and cut. The flap will then be positioned in the neck area with four stay sutures, and vascular anastomoses is performed between the femoral artery and the ECA and the femoral vein and the EJV. The skin is then closed using 6-0 nylon.

Also on Day −4, splenocytes are harvested from the auto-sensitized ACI rats and exposed in a co-culture reaction to ACI endothelial cells. On day 0, the splenocytes are isolated and assessed for apoptotic phenotypes using PI and annexin V-FITC (fluorescein) in Flow Cytometry. Ten million splenocytes are injected into the penile vein of the nude rat recipient. The nude rat is observed for 28 days and biopsies of the skin flap taken on post-operative days 0, 2, 7 and 14, or when (if) signs of rejection or Graft-versus-Host Disease (GVHD) occur. On day 28, the rats are euthanized, and samples of the skin flap, tongue, ear, liver and small bowel will be harvested. The latter serve to assess the potential development of GVHD in the nude rat recipients.

Group 2

On Day −7, WF rats are injected with 10⁷ ACI splenocytes. On Day −4, nude rats are transplanted with ACI skin flaps in the neck area. Also on Day −4, splenocytes are isolated from the allosensitized WF rats and exposed in a co-culture to ACI endothelial cells. On day 0, the splenocytes are harvested, and 107 of them injected into the penile vein of the nude rat recipient. The rats are then observed as for Group 1.

Group 3

On Day −7, WF rats are injected with 10⁷ ACI splenocytes. On Day −4, nude rats are transplanted with ACI skin flaps in the neck area. Also on Day −4, splenocytes are isolated from the allosensitized WF rats and exposed in a co-culture to ACI endothelial cells that are coated with the candidate apoptosis-inducing molecule. On day 0, the splenocytes are harvested, and 107 splenocytes injected into the penile vein of the nude rat recipient. The rats are then observed as for Group 1.

The following, not meant to limit the invention, is presented to aid the practitioner, although other methods, techniques, cells, reagents and approaches can be used.

Fusogenic Lipid Vesicles

Lipid vesicles resemble plasma membranes, and they can be made to fuse with cell membranes. Previous liposome studies have shown that four major types of interactions are observed between liposomes and cel membranes: adsorption to cell surface; endocytosis (the active taking-up of the liposome by phagocytic cells); lipid exchange (involving the transfer of individual lipid molecules between the liposome and the plasma membrane); and fusion (where the liposome membranes unite with plasma cell membranes). The interaction between lipid vesicles and cell membranes is probably similar to those between liposomes and cell membranes. Fusion provides the most attractive mechanisms since it allows for the direct introduction of vesicular contents into the cell. Adsorption or lipid exchange can occur when a vesicle is not very fusogenic and do not allow for the delivery of vesicular aqueous contents. Endocytosis can only occur in certain types of cells, such as leukocytes.

However, most liposomes and multilamellar vesicles are not readily fusogenic, mainly because the stored energy of the vesicle radius of curvature is minimal. But the small unilamellar vesicles of the present invention, which have a very tight radius of curvature, are very fusogenic. The average diameter of a small unilamellar vesicle (FUV) is 5 nm to 500 nm; preferably 10 nm to 100 nm, more preferably 20 nm to 60 nm, including 40 nm. This size allows vesicles to pass through the gaps between endothelial cells. Useful vesicles may vary greatly in size and are selected according to a specific application.

The compositions from which the vesicles of the present invention are formed contain a phospholipid which is a stable vesicle former, preferably together with another polar lipid, and optionally with one or more additional polar lipids and/or raft formers.

Polar lipids are organic molecules which have a hydrophobic end and a hydrophilic end, and contain at least six carbon atoms; they have the structure of formula (I), where X is a head group, L is a back bone group, and each Z is a fatty group. The two Z groups may be the same or different. A phospholipid is a polar lipid which has a head group of formula (II), where A and B are substituents of the head group.

The head group, X, may be any polar group, preferably a cationic, anionic or zwitterionic group, or H. More preferably X is group of formula (II). Preferably, B is an cation, such as Na⁺, K⁺, or tetramethyl ammonium ion; or an alkyl group. Preferably, A is H, or an alkyl group; more preferably A is an alkyl group substituted with an amine; most preferably A is a group of formula (III), (IV), (V), (V) or (VII). It should be noted that throughout the specification, the formulas may show the structures in protonated form, but that they also include the unprotonated form (and visa versa); which form is present in any composition will depend on the exact pH of the composition, and the presence of water and/or appropriate counter ions.

The back bone group, L, is an alkyl further missing two hydrogen atoms (to give a total of three open attachment points), preferably an alkoxy, or amino substituted alkyl. Most preferably, L is a group of formula (VIII), (IX) or (X).

The fatty groups, Z, may be the same or different, and are H, an E group, or the structure of formula (XI), where E is an alkyl or alkenyl. Preferably, E is an unsubstituted straight chain alkyl or alkenyl, with 6-26 carbon atoms; more preferably E is a group of formula (XII), (XIII), (XIV), (XV), or (XVI). If one of the fatty groups is H, then the other must be different. If double bands are present, then cis configuration is preferable.

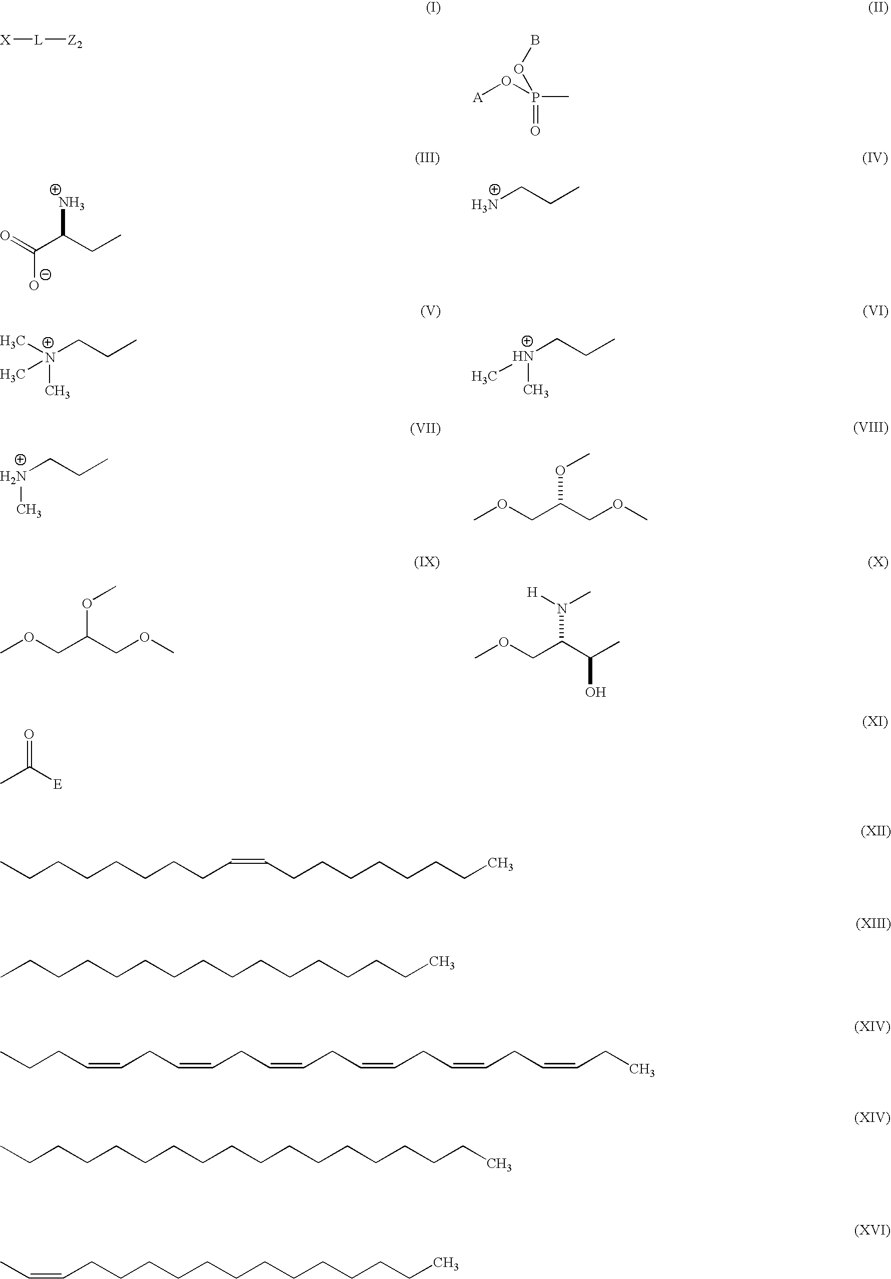

A phospholipid (or polar lipid) which is a stable vesicle former is a phospholipid (or polar lipid) that will form vesicles, at least 50% of which persist for at least one hour, when prepared as follows: the phospholipid is dissolved in chloroform and placed in glass test tube Solvent is removed by evaporation under a steady stream of nitrogen, followed by air removal by subjecting the sample to vacuum for twelve hours. The dried lipid material is then re-hydrated in 10 mM Na₂HPO₄, for 60 minutes at a temperature above the lipid phase transition temperature; the desired final concentration is 25 mg/ml. The lipid mixture is then agitated by sonication with a microtip 450 watt sonicator used at a 40% duty cycle.

Preferably, in addition to the phospholipid which is a stable vesicle former, at least one other polar lipid is included, more preferably one or more polar lipids which are not stable vesicle formers.

A raft former is a compound which will sit within the lipid layer of a vesicle when the vesicle is in an aqueous solution, and will form or cause formation of discrete regions within the vesicle wall (also known as rafts). These discrete regions tend to destabilize the vesicle, increasing its fusogenicity. Examples of raft formers are cholesterol (formula XXIV), sphingomyelin, and proteins and polypeptides know to be membrane bound. Fusogenicity may also be enhanced by selecting polar lipids, which will result in a surface charge on the vesicle, which is the opposite of the charge of the Gouey-Chapman layer of the target cells (typically the Gouey-Chapman layer is positively charged).

Examples of polar lipids for use in the present invention include 1,2-dioleoyl-sn-glycero-3-phosphocholine (DOPC) (formula XVII; a stable vesicle former), 1-palmitoyl-2-oleoyl-sn-glycero-3-phosphate (POPA) (shown as the monosodium salt in formula XVIII), 1,2-dioleoyl-sn-glycero-3-ethylphosphocholine (DOPC-e) (shown as the chloride salt in formula XIX), 1,2-dioleoyl-sn-glycero-3-phosphoethanolamine (DOPE) (formula XX), 1,2-dioleoyl-sn-glycero-3-[phospho-1-serine] (DOPS) (shown as the sodium salt in formula XXI), 1-palmitoyl-2-docosahexaenoyl-sn-glycero-3-phosphocholine (formula XXII; a stable vesicle former), a typical sphingomyelin (formula XXIII; cholesterol will form rafts when added to a vesicle formed from a mixture this sphingomyelin and DOPC), 1,2-dimyristoyl-sn-glycerol (formula XXV), and 1-palmitoyl-2-hydroxy-sn-glycero-3-phosphocholine (XXVI). Other polar lipids useful for the practice of the present invention include phosphatidyl serine (PS), phosphatidyl glycerol (PG), mixed chain phosphatidyl choline (MPC), phosphatidyl ethanol (PE), and phospholipids containing decosahexaenoic acids. Cit-DOPC and cit-DOPC-e are especially useful. Phosphatidylcholines, including those having a docosahexaenoic acid in the sn-1 and sn-2 positions (DHPC) may be used. Other diunsaturated lipids, such as diarachidonylphosphatidylcholine (for example 20:4 DOPC:DArPC), dilinolenoylphosphatidylcholine (for example 18:3 DOPC:DLnPC) are also useful. For example, DOPC may be mixed with increasing amounts of DLNPC, DArPC and DHPC during FUV preparation. Useful ratios include (DOPC:DLnPC, DArPC or DHPC) range from 1-1000:1, such as 25-500:1, including 1:1, 25:1, 50:1, 100:1, 500:1, and 1000:1. Combinations of phospholipids having large mean molecular areas can also be used, such as DOPC:DLnPC:DHPC. Diacylglyercol, a non-lamellar phase lipid, can also be mixed with DOPC. In addition, one can use polyethylene glycol (PEG) with weights of 20 repeats up to 4000 repeats.

Preferably, the ratio of the stable vesicle former phospholipid to the polar lipid which is not a stable vesicle former is 1:1 to 500:1, more preferably 10:1 to 100:1 (for example, 50:1). Examples include: DOPC/DOPC-e (1:1); DOPC/POPA (50:1) and DOPC/POPA (1:1).

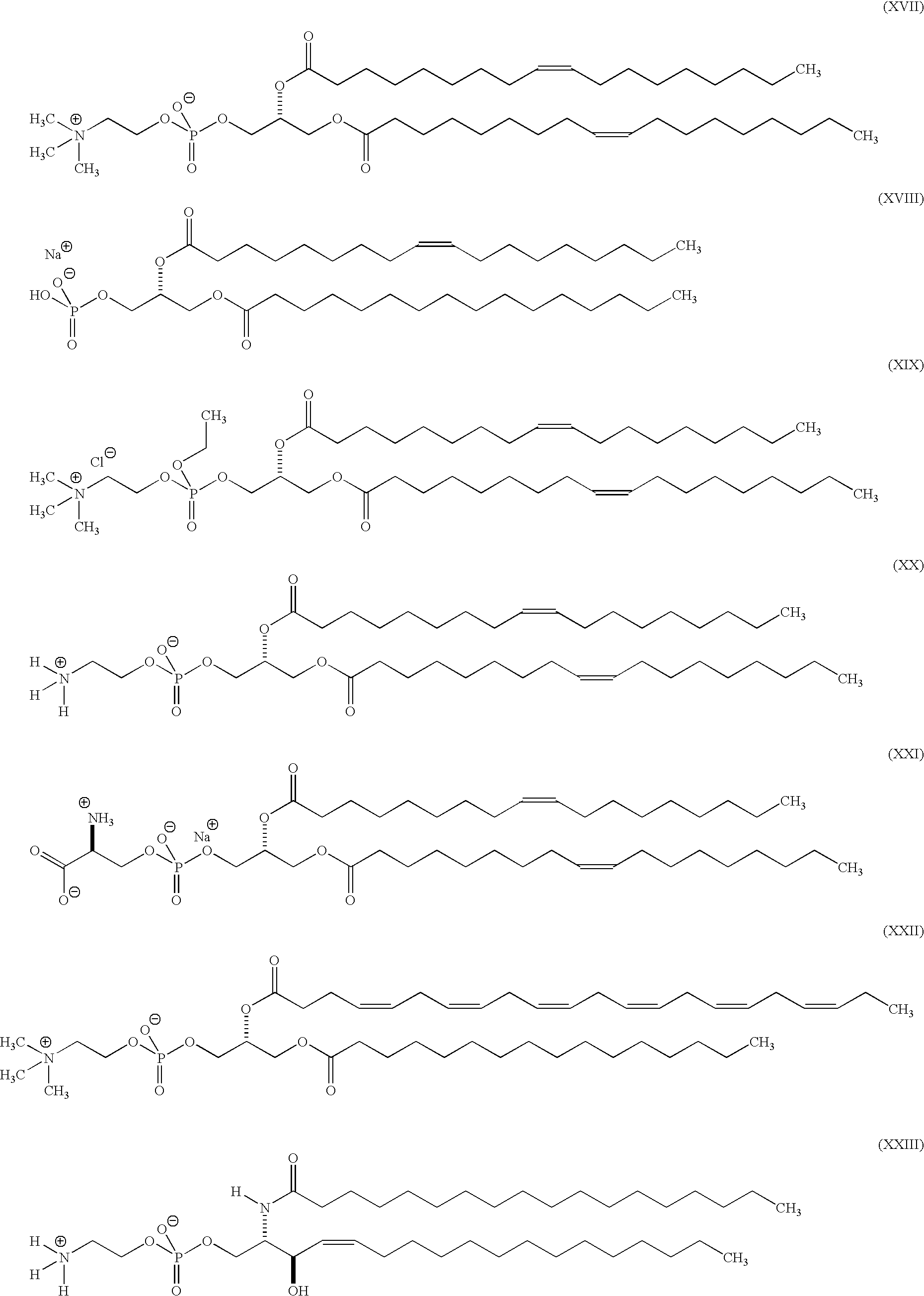

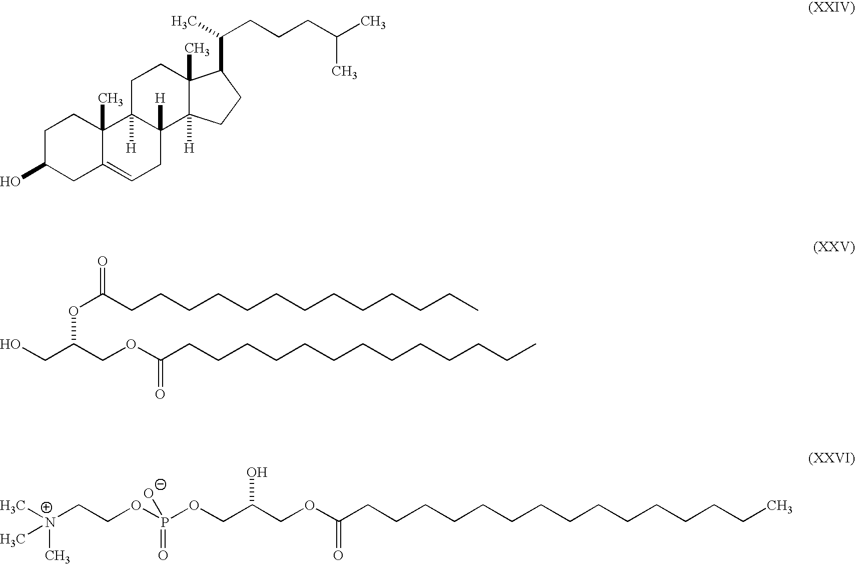

Lipid Vesicle Construction

To construct lipid vesicles, lipids are dissolved in chloroform or other appropriate organic solvent and placed in a vessel, such as glass test tube. Solvent is removed by evaporation under a steady stream of nitrogen or other neutral gas, followed by air removal, such as subjecting the sample to a vacuum for 0.1 to 48 hours, such as 1, 2, 3, 4, 5, 6, 7, 8, 9, 10, 11, 12, 15, 20, 24, 25, 30, 36, 40, 42 or 48 hours. Twelve hours usually suffices. The dried lipid material is then re-hydrated in an appropriate buffer, such as Hank's Balanced Salt Solution (HBSS) or 10 mM Na₂HPO₄, for 30-60 minutes at a temperature above the lipid phase transition temperature; the desired final concentration is usually approximately 1 to 30 mg/ml, typically around 25 mg/ml. The lipid mixture is then agitated. For example, sonication can be used; such as a microtip 450 watt sonicator used at a 40% duty cycle to create FUVs. The length of time of sonication depends on the amount of lipid material; in any case, sonication is stopped when no further decreases in percent transmission are observed or the correct vesicle size is achieved by analysis using a particle size analyzer. Lipids can be analyzed by UV spectroscopy and thin layer chromatography (TLC) to assess the extent of oxidation, if desired.

Other solutions may be used when rehydrating the dried lipids. These include those buffered with N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glyclclycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), and tris(hydroxymethyl)-aminomethane (Tris). Other examples of suitable solutions include salt solutions, such as Alseverr's Solution, Dulbecco's Phosphate Buffered Saline (DPBS), Earle's Balanced Salt Solution, Gey's Balanced Salt Solution (GBSS), Puck's Saline A, Tyrode's Salt Solution, St. Thomas Solution and University of Wisconsin Solution.

Other components may be incorporated into FUVs to manipulate their fusion rates. For example, polypeptides that are involved in membrane fusion, such as fertilin, soluble N-ethylmaleimide-sensitive factor attachment protein receptors (SNAREs), SM (sec1/munc18) polypeptides (such as mammalian isoforms of Vps33p, Sly1p and Vps45p; cit_bf(Jahn and Sudhof 1999)cit_af ref_bf(Jahn, R. 1999 ref_num30)ref_af) and viral envelope fusion proteins, such as those from Human Immunodeficiency Virus (HIV; e.g., gp41), Semiliki Forest virus, and Influenza). The mammalian SNARE family includes the syntaxins (1A, 1B, 1C; 2 (and splicing variants); 3, 3A, 3B, 3C, 3D; 4; 5, 5A, 5B, 6, 7, 8, 10, 11, 12, 13 (may be identical to 12); 16 (A, B, C); and 17), Hsyn 16, rbet1, GS15, GOS32, GOS28, Membrin, the SNAPs (25, 25a, 25b; 23, 23A, 23B; 29), vti1b, Synaptobrevins (1 and splicing variants; 2), Cellubrevin, VAMP4, VAMP5/6, Ti-VAMP, Endobrevin, Tomosyn and msec22b cit_bf(Jahn and Sudhof 1999)cit_af ref_bf(Jahn, R. 1999 ref_num30)ref_af. Other amphiphilic peptides that destabilize membranes, even if their primary function is not to mediate membrane fusion, can also be used to promote fusion, such as annexins cit_bf(Jahn and Sudhof 1999)cit_af ref_bf(Jahn, R. 1999 ref_num30)ref_af.

The methods of making the vesicles do not usually require modification when biotinylated lipids are also incorporated.

To target specific cells, polypeptides that either interact with a polypeptide specific to the targeted cell, such as a ligand-receptor interaction (at least in the area in which the FUVs are administered), or antibodies recognizing cell-specific antigens may be incorporated into FUVs. Other targeting polypeptides include those used during intercellular membrane transport and the Rab GTPase proteins. Viral fusion proteins can also be exploited as targeting molecules. Membrane bound substances, such as biotinylted lipids, and carbohydrates may also be used.

ATP Encapsulation

Typically, the magnesium salt of ATP is added at the time of lipid re-hydration. ATP concentration may vary and will depend on the application. Concentrations of ATP that are preferably used include 0.01 mM to 200 mM, preferably 0.1 mM, 1 mM, 2.5 mM, 5 mM, 7.5 mM, 10 mM, 25 mM, and 50 mM, and more preferably, 0.1 mM, 1 mM, 10 mM. The buffer containing the ATP should have a low protein content to decrease the chance of non-specific absorption of the lipid material. FUVs that contain ATP are referred to as ATP-FUV for convenience.

Encapsulation of ATP by FUVs can easily be assessed. For example, labeled ATP molecules (such that the label does not interfere with vesicle formation), such as radiolabeled ATP, preferably tritiated ATP is used. Radiolabels include ³²P, and ³H and are added when the lipids are re-hydrated after drying, prior to agitation. The solution is applied to a Sephadex G-25 column (or other suitable matrix) to remove non-encapsulated ATP. The effluent from the column is collected and assayed for the presence of vesicles. FUVs are usually eluted in the earliest fractions. Percent encapsulation is determined by quantifying the radioactivity in the vesicle and supernatant fractions, and determining the proportion of encapsulated ATP and multiplying by 100. Preferable encapsulation percentages range from approximately 1% to 10%.

Molecules other than ATP may be delivered to cells using FUVs, such as organic and inorganic molecules, including pharmaceuticals, polypeptides, nucleic acids and antibodies that interact with intracellular antigens.

Assay for Measuring FUV Fusogenicity

The fusion rate is a measure of the number of lipid vesicles that fuse with the HUVEC cells in a well/second (about 10⁶ cells), the assays has the following steps:

(1) HUVEC cells (American Type Culture Collection (ATCC); Manassus, Va. or BioWhittaker; MD) are cultured;

(2) FUVs are prepared and loaded with a fluorescent probe, such as carboxyfluorescein;

(3) the FUVs are contacted to the cells to allow for fusion;

(4) at a selected time, any residual FUVs are removed; and

(5) fluorescence is measured.

The presence and intensity of a fluorescent signal after removing the FUVs indicates the ability of the FUVs to fuse with the cell membranes and deliver the contents.

Human umbilical vein endothelial cells (HUVECs) is given as an example. The cells are grown to confluence on a standard 12-well culture dishes (for example, from COSTAR; the number of cells is approximately 106) in endothelial cell growth medium (EGM). The HUVECs are then washed 3 times with a buffer, such as HBSS. Prepared lipid vesicles (such as DOPC/DOPC-e (1:1); DOPC/POPA (50:1), DOPC/POPA (1:1), PS, PG, MPC, PE, cit-DOPC and cit-DOPCe), are loaded with 1 mM carboxyfluorescein. The vesicles are incubated with the cells for 120 minutes, assaying fluorescence at each 5 minute increment, at 37° C., 95% air/5% CO₂, after which time residual vesicles are removed by washing the cells with buffer. If negatively charged lipid vesicles are used, calcium (final concentration 0.1-10 mM) is added at the fusion step.

Cells are removed from the dish by treating with trypsin. Fluorescence is measured (excitation at 495 nm and emission of 520 nm) using a luminescence spectrophotometer or other suitable device.

The rate of fusion for ATP-FUV compositions is approximately 20 vesicle fusions/second to 8.0×10¹¹ vesicle fusions/second, including 500 to 1×10⁸ vesicles fusions; 750,000 to 50×10⁷ vesicle fusion/second; 5×10⁶ to 1×10⁷ vesicle fusions/second; including 1×10⁶ to 8×10⁸ vesicle fusions/second; 1×10⁷ to 5×10⁸ vesicle fusions/second; and 5×10⁷ to 1×10⁸ vesicle fusions/second. Examples of fusion rates are at least 100, 1000, 10⁴, 10⁵, 10⁶, 10⁷, 10⁸, 10⁹, 10¹⁰, and 10¹¹ vesicle fusions/second. Some of these values were obtained experimentally at 37° C. using mixtures of DOPC and DOPC/DOPC-e and DOPC/POPA, with and without calcium, and using human endothelial cells.

Because the lipid composition of plasma membranes varies by cell type, the choice of cells for use in the assay is carefully considered, and should match as best the target cell type(s). For example, liver cell plasma membranes consist of about 7% phosphatidylethanolamine, while red blood cell plasma membranes contain 18% cit_bf(Alberts et al. 2002)cit_af ref_bf(Alberts, B. 2002 ref_num32)ref_af. Primary culture cells, as well as cell lines (available from the American Type Tissue Collection (ATCC); Manassus, Va.) are useful, although primary cultures are preferred because of the likelihood that the plasma membrane lipid composition is altered in transformed cells. Cell types include pancreas, intestinal, immune system, neuronal (including those of the brain, eye, nose and ear), lung, heart, blood, circulatory (lymph and blood), bone, cartilage, reproductive, glandular, enamel, adipose, skin, and hepatic. Cell lines include those derived from these tissues, such as Madin-Darby canine kidney (MDCK), Chinese hamster ovary (CHO), HeLa, etc. Cells may be from other organisms, such as plants, fungi (including yeasts), and bacteria. Examples of fusion rates with these other cell types include at least 100, 1000, 10⁴, 10⁵, 10⁶, 10⁷, 10⁸, 10⁹, 10¹⁰, and 10¹¹ vesicle fusions/second. Unless otherwise specified, fusion rates are with respects to HUVECs under the conditions specified above. Fusion rates with respects to other cell types is for about 10⁶ cell, with a buffer, such as HBSS, and the vesicles are incubated with the cells for 120 minutes at 37° C., 95% air/5% CO₂, after which time residual vesicles are removed by washing the cells with buffer.

Assays for Optimizing Fusion Rates

The assay for fusion rate can be further modified when optimizing the fusion rate of a particular vesicle composition with a particular cell type. For example, the lipid vesicle can contain a fluorescent or radioactive tracer that is part of the membrane bilayer of the vesicle.

Other fluorescent probes may also be used. These include fluorescein isothiocyanate; fluorescein dichlorotriazine and fluorinated analogs of fluorescein; naphthofluorescein carboxylic acid and its succinimidyl ester; carboxyrhodamine 6G; pyridyloxazole derivatives; Cy2, 3 and 5; phycoerythrin; fluorescent species of succinimidyl esters, carboxylic acids, isothiocyanates, sulfonyl chlorides, and dansyl chlorides, including propionic acid succinimidyl esters, and pentanoic acid succinimidyl esters; succinimidyl esters of carboxytetramethylrhodamine; rhodamine Red-X succinimidyl ester; Texas Red sulfonyl chloride; Texas Red-X succinimidyl ester; Texas Red-X sodium tetrafluorophenol ester; Red-X; Texas Red dyes; tetramethylrhodamine; lissamine rhodamine B; tetramethylrhodamine; tetramethylrhodamine isothiocyanate; naphthofluoresceins; coumarin derivatives; pyrenes; pyridyloxazole derivatives; dapoxyl dyes; Cascade Blue and Yellow dyes; benzofuran isothiocyanates; sodium tetrafluorophenols; and 4,4-difliuoro-4-bora-3a,4a-diaza-s-indacene. The excitation wavelength will vary for these compounds. Lipid vesicles are made in the presence of the tracer in ratios such as 1:800 lipid/probe. Other useful ratios include 1:200 to 1:10,000, including 1:400, 1:500, 1:600, 1:700, 1:800, 1:900 and 1:1000.

Altering Fusion Rates

The fusion rate of any lipid vesicle can be altered by changing a variety of factors, such as temperature, ions, lipid concentration, lipid vesicle composition, flow rates, lipid vesicle size, etc. Altering the phospholipid formulation of FUVs can be used to maximize fusion rates as well as minimize toxicity. For example, to preserve organs for transplant or cells in suspension (such as blood), FUVs that have slower, delayed fusion rates are desirable. Such rates are obtained with vesicles consisting of only DOPC. On the other hand, if immediate raising of the intracellular ATP is crucial, as for stroke, heart attack or trauma sufferers, FUVs with very fast rates of delivery are desirable; DOPC/POPA compositions, for example, deliver sufficient ATP in less than five minutes (see Examples).

Four general approaches can be used to alter fusion rates by manipulating lipid composition:

(1) increasing electrostatic interactions;

(2) destabilizing membrane bilayers;

(3) increasing non-bilayer phases; and

(4) creating dissimilar lipid phases.

Increasing Electrostatic Interactions

Electrostatic interactions can be exploited to increase fusion rates. Phospholipids are classified according to their charge (cationic, anionic, and zwitterionic). Many of the cationic phospholipids, such as PE, and anionic phospholipids, such as phosphatidic acid (POPA), do not form closed vesicles at physiologic pH. However, anionic and cationic lipids mixed with zwitterionic phosphatidylcholines can form closed vesicles at physiologic pH.

The plasma membrane of most cells has a net negative charge. Because of this negative charge, there is a layer of counterbalancing ions, typically calcium, magnesium, sodium and potassium, which presents a net positive charge. Taking advantage of the electrostatic interaction between liposomes and plasma membranes, FUVs are engineered to have a net negative charge, thus maximizing cell-lipid vesicle fusion. However, some cell plasma membranes contain more cationic lipids which are counterbalanced by a anionic ion layer. In these situations, FUVs are engineered to have a net positive charge to maximize cell-lipid fusion.

Creating Dissimilar Lipid Phases

Plasma membranes contain lipid domains or rafts that are enriched in a particular lipid species. At the boundary of such a membrane raft are regions of dissimilar lipid species. These regions have the potential for instability, effecting how the membrane interacts with other membranes. Several phospholipids are known to increase lipid raft formation, including mixtures of phosphatidylcholines, sphingomyelin, and cholesterol. For example, DOPC, 18:0 sphingomyelin, and cholesterol are mixed in a 1:1:1 ratio during FUV preparation. Cholesterol preferentially partitions in the sphingomyelin phase, creating regions that are rich in DOPC and poor in cholesterol, and regions that are rich in sphingomyelin and rich in cholesterol.

Changing the physical parameters of fusion, temperature, concentration, ionic strength, and fusion period, can be used to affect fusion rates. By altering temperature, the free energy (G) of the system is altered, leading to different rates of fusion. Increasing lipid vesicle concentration also affects membrane fusion rates, especially at very high concentrations. The fusion period (length of fusion) and the number of fusion periods also affect the rate of delivery of the encapsulated contents of FUVs.

Temperature

ATP-FUV is incubated with tissues 5, 10, 15, 30, 60 or 120 minutes at the temperatures at which the tissues are being preserved (4° C.-hypothermia, 22° C.-room temperature, 37° C.-normothermia). Increasing the temperature of the vesicle solution leads to increased kinetic energy of the vesicles and hence increased capability to fuse. Temperature also affects the free diffusion of the vesicles.

Concentration on Vesicle Fusion

While intuitive that increased concentration leads to increased FUV content delivery, the rate of membrane fusion is not linear. Once FUV lipids occupy all of the available plasma membrane surface, further fusion is limited. The extent of fusion with the plasma membrane affects membrane volume and properties, such as ion permeability and lipid organization. Therefore, when administering FUVs, FUV concentration must be controlled so that the target cells are effectively treated.

Fusion Period

The length of time that fusion is allowed to occur helps to control the extent to which encapsulated substances are delivered. Preferable fusion periods are 1-180 minutes, such as 1, 5, 10, 30, 60, 120 and 180 minutes. To halt fusion, the vesicles are removed (such as by washing with a buffer), or the concentration of the administered vesicles is such that the vesicles are depleted at the end point of the desired time. Fusion may also be optimized such that the total delivery of the vesicles is controlled through one or multiple administrations. For example, if the target fusion period is 120 minutes, two 60 minute periods may be used, or four 30 minute, twelve 10 minute, or 24 five minute fusion periods. Provided that proper equipment is available, 1 minute or less fusion periods may also be accomplished, although these periods are often inconvenient and technically demanding.

Determining ATP Requirements of the Targeted Cells and Tissues

The optimum rate of ATP administration is that which approximates the basal metabolic demand for ATP of cells; this can be determined by any method known in the art. Oxygen consumption rates, pyruvate, glucose, lactate, and proton leak can be calculated, and from this data, the ATP consumption of the tissues is determined as ATP consumed/minute.

Tissue Oxygen Consumption

Samples of tissue are placed in a pre-cooled to −20° C. glass homogenizer. Ice cold isolation buffer, such as 200 mM sucrose, 70 mM KCl, 5 mM maleate and 40 mM Tris at pH 7.3, is added, and the tissue gently homogenized. The homogenate is briefly centrifuged to remove non-homogenized material. Five milliliters of oxygenation buffer is then placed in an oxygen meter and allowed to equilibrate to 37° C. The cells are placed in a YSI oxygen bath stirrer (Yellow Springs, Ohio) to a final protein concentration of 2-3 mg/ml. An oxygen probe is placed into the solution, and a YSI oxygen meter is used to measure the % oxygen in the solution. ADP is then added to the bath to achieve State 2 respiration rate, followed by glutamate addition, achieving State 3 respiration rate. Once the glutamate is consumed by the tissue, a final state of respiration is achieved, State 4. A plot of the State 3 respiration rate versus the amount of ADP that was added to the homogenate allows for the calculation of the phosphorus/oxygen (P/O) ratio. This value determines the amount of ATP the tissue can produce from ADP/minute, which is an index of the amount of ATP the tissue consumes/minute.

Membrane Potential and Proton Leak

Tissue samples are isolated and incubated with the membrane potential fluorescent probe MC540 (Sigma; St. Louis, Mo.). Changes in fluorescence of MC540 upon addition of various amounts of potassium is measured as an indice of membrane potential and proton leak as previously described (Brand, 1995).

Glucose, Pyruvate, and Lactate Levels

These metabolic intermediates are determined using standard methods or commercially-available analysis kits (such as those available from Sigma). The levels of these intermediates are adjusted to protein levels and are measured over a 120 minute time period.

Determination of ATP Consumption

From the rates of lactate, pyruvate, and glucose accumulation, oxygen consumption, and proton leak, it is possible to calculate all of the fluxes through the system by using reaction stoichiometries as described by Ainscow and Brand (1999).

Administration

Pharmaceutical Compositions

In many cases, ATP-FUV may be delivered as a simple composition comprising the ATP-FUV and the buffer with which it was made. However, other products may be added, if desired, such as those traditionally used as carriers in pharmaceutical compositions.

A “pharmaceutically acceptable carrier” includes any and all solvents, dispersion media, coatings, antibacterial and antifungal agents, isotonic and absorption delaying agents, and the like, compatible with pharmaceutical administration (Remington 2000). Preferred examples of such carriers or diluents include water, saline, Ringer's solutions and dextrose solution. Supplementary active compounds can also be incorporated into the compositions.

General Considerations

A pharmaceutical composition of the invention is formulated to be compatible with its intended route of administration, including intravenous, intradermal, subcutaneous, oral, inhalation, transdermal, transmucosal, and rectal administration. Solutions and suspensions used for parenteral, intradermal or subcutaneous application can include a sterile diluent, such as water for injection, saline solution, polyethylene glycols, glycerine, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

If negatively charged lipid vesicles are used in the ATP-FUV compositions, calcium is included such that the final concentration at the site of fusion is preferably 0.1 mM-10 mM; including 0.1, 1, 2, 3, 4, 5, 6, 7, 8, 9 or 10 mM.

The ATP in ATP-FUVs is usually in equilibrium with the ATP in any solution surrounding the ATP-FUVs; typically only 1-10% of the total ATP is within the ATP-FUVs. The remaining ATP may bind to receptors, such as the purinoreceptor P2y, causing ions to flow out of the cells, and interfering with ion balance and homeostasis. Although the cells can usually reestablish ion balance and homeostasis, this consumes additional ATP. Therefore, particularly with tissue for which immediate restoration of function is desirable (for example, during organ transplantation, or limb reattachment), including in the composition one or more purinoreceptor P2y antagonists, is advantageous. The purinoreceptor P2y antagonists is preferably added to the composition after forming the vesicles, or just prior to administration, since the antagonists do not need to be within the FUVs. Examples of purinoreceptor P2y antagonists include pyridoxal 5-phoshpate, vitamin B6 (pyridoxal-5-phosphoric acid), and Reactive Blue 2 (1-amino-4-[[4-[[4-chloro-6-[[3(or 4)-sulfophenyl]amino]-1,3,5-triazin-2-yl]amino]-3-sulfophenyl]amino-9, 10-dihydro-9,10-dioxo-2-anthracenesulfonic acid), and combinations thereof. The purinoreceptor P2y antagonists may preferably be used in a concentration of 0.1 to 250 micromoles/L, more preferably 1-100 micromoles/L.

Injectable Formulations

Pharmaceutical compositions suitable for injection include sterile aqueous solutions or dispersions for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a dispersion medium containing, for example, water, polyol (such as glycerol, propylene glycol, and liquid polyethylene glycol), and other compatible, suitable mixtures. Various antibacterial and anti-fungal agents, for example, parabens, chlorobutanol, phenol, ascorbic acid, and thimerosal, can contain microorganism contamination. Isotonic agents such as sugars, polyalcohols, such as manitol, sorbitol, and sodium chloride can be included in the composition. Compositions that can delay absorption include agents such as aluminum monostearate and gelatin.

Sterile injectable solutions can be prepared by incorporating ATP-FUV in the required amount in an appropriate solvent with one or a combination of ingredients as required, followed by sterilization. Methods of preparation of sterile solids for the preparation of sterile injectable solutions include vacuum drying and freeze-drying to yield a solid containing ATP-FUV lipids and any desired ingredient (such as ATP) a sterile solutions.

Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

Compositions for Inhalation

For administration by inhalation, the compounds are delivered as an aerosol spray from a nebulizer or a pressurized container that contains a suitable propellant, e.g., a gas such as carbon dioxide.

Transmucosal or Transdermal

Administration can be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams. Suppositories (e.g., with bases such as cocoa butter and other glycerides) or retention enemas for rectal delivery may also be prepared.

Carriers

In one embodiment, the active compounds are prepared with carriers that protect the compound against rapid elimination from the body, such as a controlled release formulation, including implants and microencapsulated delivery systems. Biodegradable or biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art.

Dosage

Dosage is dictated by, and directly depends on, the unique characteristics of ATP-FUV which varies with different FUV lipid compositions, the particular desired therapeutic effect, and the route of administration. The specific dose level and frequency for any particular patient or application may be varied. Factors that should be considered, including (1) the temperature at which administration is made and at which fusion is permitted; (2) the ionic environment of the administration site and the ionic strength of the ATP-FUV composition; and (3) the length of time that fusion is permitted. Controlling these factors helps to control the extent to which the encapsulated substances, including ATP, are delivered.

When administering FUVs, FUV concentration is controlled to effectively treat the target cells while not inhibiting their function by saturating the plasma membranes with FUV lipids. Preferable concentrations of FUV, depending on lipid composition, target cell dispersion and volume to be administered may be 0.5 mg/ml-100 mg/ml, such as 0.5 mg/ml, 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml and 100 mg/ml.

Vesicle fusion occurring via electrostatic interactions is significantly affected by changes in calcium and/or magnesium concentrations, and to a lesser extent, changes in sodium and/or potassium concentrations. Modulating these ion concentrations either in the compositions used to administer ATP-FUV or in compositions administered to a target site before or after ATP-FUV administration, affect dosage considerations. Preferably, ion concentrations of 0.01 nM to 1 mM, including 0.1 nM, 1 nM, 10 nM, 100 nM, 1000 nM, 10 micromole/L, and 100 micromoles/L are used. Combinations of these and other ions may also be used.

Regimes of chronic administration or single dosing can be used and are chosen according to the type of treatment, administration route, the vesicles themselves. Preferable fusion periods include 1-180 minutes, such as 1, 5, 10, 30, 60, 120 and 180 minutes. To halt fusion, the ATP-FUV is removed (such as by washing with a buffer), or the concentration of vesicles is such that the vesicles are depleted at the end point of the desired time. Fusion can also be optimized such that the total delivery of the vesicles is controlled through one or multiple administrations. For example, if the fusion period is 120 minutes, two 60 minute periods may be used, or four 30 minute periods, twelve 10 minute periods, or 24 five minute fusion periods.

Creating FUVs Containing Lipids

In the case of biotinylated lipids, FUVs composed of DOPC/POPA and biotinylated lipids (N-biotinoyl-1,2-dihexadecanoyl-sn-glycero-3-phosphoethanolamine (BDGP; Molecular Probes; Eugene, Oreg.) at a preferred ratio of 1:5, BDGP:DOPC phospholipids are produced. The ratio of biotinylated phospholipids can range from 1:0 BDGP:DOPC, 1:1, 1:2, 1:3, 1:4, 1:5, 1:6, 1:7, 1:8, 1:9, 1:10, 1:15, 1:20, 1:25, 1:30, 1:40, 1:50, 1:75, 1:100, 1:250, 1:500, 1:750, 1:1,000 and 1:10,000 depending on the target cell type. For endothelial cells, however, the ratio of 1:5 is preferred.

Other biotinylated lipids may be used, again depending on the target cell type. Such lipids are selected based on the desired composition of the FUVs and are either obtained commercially or synthesized according to established protocols (Avila-Sakar and Chiu, 1996; Kuhl et al., 1998; Rivnay et al., 1987).

Similar ratios may be applied to lipids derivatized with other tethers, such as, for example, FITC. When the apoptosis-inducing molecule is incorporated directly into the FUV, then molar ratios are experimentally determined by established protocols.

Determining ATP Requirements of the Targeted Cells and Tissues

The optimum rate of ATP administration is that which approximates the metabolic demand for ATP of cells; this can be determined by any method known in the art. Oxygen consumption rates, pyruvate, glucose, lactate, and proton leak can be calculated, and from this data, the ATP consumption of the tissues is determined as ATP consumed/minute.

Tissue Oxygen Consumption

Samples of tissue are placed in a pre-cooled to −20° C. glass homogenizer. Ice cold isolation buffer, such as 200 mM sucrose, 70 mM KCl, 5 mM maleate and 40 mM Tris at pH 7.3, is added, and the tissue gently homogenized. The homogenate is briefly centrifuged to remove non-homegenized material. Five milliliters of oxygenation buffer is then placed in an oxygen meter and allowed to equilibrate to 37° C. The cells are placed in a YSI oxygen bath stirrer (Yellow Springs, Ohio) to a final polypeptide concentration of 2-3 mg/ml. An oxygen probe is placed into the solution, and a YSI oxygen meter is used to measure the % oxygen in the solution (State 1). ADP is then added to the bath to achieve State 2 respiration rate, followed by glutamate addition, achieving State 3 respiration rate. Once the glutamate is consumed by the tissue, a final state of respiration is achieved, State 4. A plot of the State 3 respiration rate versus the amount of ADP that was added to the homgenate allows for the calculation of the phosphorus/oxygen (P/O) ratio. This value determines the amount of ATP the tissue can produce from ADP/minute, which is an index of the amount of ATP the tissue consumes/minute.

Membrane Potential and Proton Leak

Tissue samples are isolated and incubated with the membrane potential fluorescent probe MC540 (Sigma; St. Louis, Mo.). Changes in fluorescence of MC540 upon addition of various amounts of potassium is measured as an indice of membrane potential and proton leak as previously described (Brand, 1995).

Glucose, Pyruvate, and Lactate Levels

These metabolic intermediates are determined using standard methods or commercially-available analysis kits (such as those available from Sigma). The levels of these intermediates are adjusted to polypeptide levels and are measured over a 120 minute time period.

Determination of ATP Consumption

From the rates of lactate, pyruvate, and glucose accumulation, oxygen consumption, and proton leak, it is possible to calculate all of the fluxes through the system by using reaction stoichiometries as described by (Ainscow and Brand, 1999).

Administration

Pharmaceutical Compositions

In many cases, A-FUV may be delivered as a simple composition comprising the A-FUV and the buffer with which it was made. However, other products may be added, if desired, such as those traditionally used as carriers in pharmaceutical compositions.

A “pharmaceutically acceptable carrier” includes any and all solvents, dispersion media, coatings, antibacterial and antifungal agents, isotonic and absorption delaying agents, and the like, compatible with pharmaceutical administration cit_bf(Anonymous 2000)cit_af ref_bf(2000 ref num200)ref_af. Preferred examples of such carriers or diluents include water, saline, Finger's solutions and dextrose solution. Supplementary active compounds can also be incorporated into the compositions.

General Considerations

A pharmaceutical composition of the invention is formulated to be compatible with its intended route of administration, including intravenous, intradermal, subcutaneous, oral (e.g., inhalation), transdermal (i.e., topical), transmucosal, and rectal administration. Solutions and suspensions used for parenteral, intradermal or subcutaneous application can include a sterile diluent, such as water for injection, saline solution, polyethylene glycols, glycerine, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

If negatively charged lipid vesicles are used in the A-FUV compositions, calcium is included such that the final concentration at the site of fusion is 0.1 mM-10 mM; including 01, 1, 2, 3, 4, 5, 6, 7, 8, 9 or 10 mM.

Injectable Formulations

Pharmaceutical compositions suitable for injection include sterile aqueous solutions or dispersions for the extemporaneous preparation of sterile injectable solutions or dispersion. For intravenous administration, suitable carriers include physiological saline, bacteriostatic water, CREMOPHOR EL (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a dispersion medium containing, for example, water, polyol (such as glycerol, propylene glycol, and liquid polyethylene glycol), and other compatible, suitable mixtures. Various antibacterial and anti-fungal agents, for example, parabens, chlorobutanol, phenol, ascorbic acid, and thimerosal, can contain microorganism contamination. Isotonic agents such as sugars, polyalcohols, such as manitol, sorbitol, and sodium chloride can be included in the composition. Compositions that can delay absorption include agents such as aluminum monostearate and gelatin.

Sterile injectable solutions can be prepared by incorporating A-FUV in the required amount in an appropriate solvent with one or a combination of ingredients as required, followed by sterilization. Methods of preparation of sterile solids for the preparation of sterile injectable solutions include vacuum drying and freeze-drying to yield a solid containing A-FUV lipids and any desired ingredient (such as ATP) a sterile solutions.

Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

Compositions for Inhalation

For administration by inhalation, the compounds are delivered as an aerosol spray from a nebulizer or a pressurized container that contains a suitable propellant, e.g., a gas such as carbon dioxide.

Systemic Administration

Systemic administration can be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams. Suppositories (e.g., with bases such as cocoa butter and other glycerides) or retention enemas for rectal delivery may also be prepared.

Carriers

In one embodiment, the active compounds are prepared with carriers that protect the compound against rapid elimination from the body, such as a controlled release formulation, including implants and microencapsulated delivery systems. Biodegradable or biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art.

Dosage

Dosage is dictated by, and directly depends on, the unique characteristics of A-FUV which varies with different FUV lipid compositions, the particular desired therapeutic effect, and the route of administration. The specific dose level and frequency for any particular patient or application may be varied and will depend upon a variety of factors, including the metabolic stability and length of action of A-FUV, the age, body weight, general health, gender, diet, mode and time of administration, rate of excretion, drug combination, the severity of the particular condition, and the host undergoing therapy. Additional factors should be considered as well, including (1) the temperature at which administration is made and at which fusion is permitted; (2) the ionic environment of the administration site and the ionic strength of the A-FUV composition; and (3) the length of time that fusion is permitted. Controlling these factors helps to control the extent to which the encapsulated substances, including ATP, are delivered.

When administering FUVs, FUV concentration is controlled to effectively treat the target cells while not inhibiting their function by saturating the plasma membranes with FUV lipids. Useful concentrations of FUV, depending on lipid composition, target cell dispersion and volume to be administered may be 0.5 mg/ml-100 mg/ml, such as 0.5 mg/ml, 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml and 100 mg/ml.

Vesicle fusion occurring via electrostatic interactions is significantly affected by changes in calcium and/or magnesium concentrations, and to a lesser extent, changes in sodium and/or potassium concentrations. Modulating these ion concentrations either in the compositions used to administer A-FUV or in compositions administered to a target site before or after A-FUV administration, affect dosage considerations. Ion concentrations of 0.01 nM to 1 mM, preferably 0.1 nM, 1 nM, 10 nM, 100 nM, 1000 nM, 10 μM, and 100 μM are used. Combinations of these and other ions may also be used.

Regimes of chronic administration or single dosing can be used and are chosen according to the type of treatment, administration route, the vesicles themselves. Typical fusion periods include 1-180 minutes, preferably 1, 5, 10, 30, 60, 120 and 180 minutes. To halt fusion, the A-FUV is removed (such as by washing with a buffer), or the concentration of vesicles is such that the vesicles are depleted at the end point of the desired time. Fusion can also be optimized such that the total delivery of the vesicles is controlled through one or multiple administrations. For example, if the fusion period is 120 minutes, two 60 minute periods may be used, or four 30 minute periods, twelve 10 minute periods, or 24 five minute fusion periods. Provided that proper equipment is available, 1 minute fusion periods may also be accomplished, although these periods are inconvenient and often technically not feasible.

Sustaining Transplants

The success of transplantation depends in large part on the ability of the transplant to survive apart from its original owner or intended recipient. The more ischemia, the less likelihood that transplantation results in a functional transplant, or even success of any kind at all.

To extend the life of a transplant and reduce ischemic injury, A-FUVs are administered; these vesicles may also incorporate the tethers intended for apoptosis-inducing molecules, or incorporate in and of themselves, the apoptosis-inducing molecules. If possible, a major feed artery of a transplant is cannulated for perfusion. The transplant is perfused with the A-FUV every 4 hours, or as determined necessary due to changes in tissue ATP levels. The arterial pressure of the transplant, if possible, is monitored during perfusion to decrease the chance of flow-induced injury, and to monitor the overall preservation of the transplant-higher perfusion pressures may indicate transplant morbidity. Following the preservation period, the transplant is flushed with Ringer's or other suitable solution to remove traces of A-FUV. The transplant is then surgically transplanted using well-known methods. External indices of transplant function after anastomoses are evaluated (color, evidence of microthrombi, coagulation, temperature, organ function) to monitor success. Prior to and following transplantation, heparin is applied and antibiotic therapy is commenced to reduce the likelihood of infection.

For example, hearts which are in full arrest or suffering from hypoxia are administered A-FUV intravenously to help offset ischemic damage. The A-FUV is injected into the heart immediately or as soon as possible following the hypoxic episode. The FUV lipid compositions are manipulated so that ATP delivery is carefully matched to the metabolic demand of heart tissue, maximizing heart performance. A-FUV may be constantly perfused into the heart at physiologic conditions until such time the danger of ischemia has passed.

In another example of organ preservation, organs (e.g., hearts, liver, lungs, kidney, pancreas or spleen) are removed from the donor, and the major feed artery into the organ is cannulated. The blood in the organ is flushed from the organ using saline, Ringers or other suitable solution. A-FUV is gently perfused (□80 mm Hg) into the organ, the frequency of which will depend on the organ. The amount of A-FUV and the exact lipid composition is determined by the skilled artisan, and will vary with the organ to be preserved. After perfusion with A-FUV and rinsing, the organ is ready for transplantation.

In another example, a Lagendorff heart (or other organ) perfusion apparatus is used. The heart main artery is cannulated an the heart is placed into a perfusion chamber. The heart is perfused with an oxygenated perfusate and pharmaceutical compositions, such as A-FUV are injected into the chamber. To inject the A-FUV, the perfusate is stopped, and the A-FUV injected. In the case of a beating heart, a high potassium solution is injected to cause cardiac arrest. The A-FUV is kept in the heart for 120 minutes at 37° C. under no-flow conditions. The heart is then flushed with oxygenated perfusate solution. Heart performance may be monitored before transplantation.

### EXAMPLES

The following examples are provided to illustrate the invention. Those skilled in the art can readily make insignificant variations in the compositions and methods of this invention. The examples are not meant to limit the invention in any way.

### Example 1

### Construction of Lipid Vesicles

This example demonstrates the construction of FUVs.

Vesicles were constructed from 1,2-dioleoyl-sn-glycero-3-phosphocholine (DOPC); 1,2-dioleoly-sn-glycero-3-ethylphosphocholine (DOPC-e) and 1-palmitoyl-2-oleoyl-sn-glycero-3-phosphate (POPA) lipids. (all from Avanti Polar Lipids; Alabaster, Ala.). The lipids were used without further purification. After dissolving the lipids in chloroform and placed in a glass test tube, the chloroform was removed by evaporation under a steady stream of nitrogen gas, followed by overnight vacuum pumping. The lipid material was hydrated in buffer solution (Ringer's lactate or modified Krebs Henseleit) to a concentration of 25 mg/ml. The buffer and lipid were mixed with a vortex for 1 minute to create multi-lamellar vesicles and then placed in a 37° C. bath for 5 minutes; this procedure was repeated 6 times. The multi-lamellar vesicles were then placed in an ice bath and subjected to pulse sonication (40% duty cycle) for 5 minutes. The resultant small, fusogenic unilamellar vesicles (FUVs) were briefly centrifuged to remove any traces of contaminating titanium from the sonicator probe. After FUVs were prepared, a sample was taken to measure the average hydrodynamic radius of FUVs using a Proterion DynaPro LSD Particle Size Analyzer (Proterion Corp.; Piscataway, N.J.) to insure uniform vesicle size.

### Example 2

### Rate of Fusion of Vesicles to HUVEC and Release of Encapsulated Contents Into the Cytoplasm

To determine the fusogenic rate of FUVs, FUVs were loaded with a fluorescent probe, presented to cells in vitro, washed, and then analyzed for cellular fluorescence.

Human umbilical vein endothelial cells (HUVEC) were purchased from BioWhitaker (Walkersville, Md.) at passage 1 and cultured until passage 8, after which they were no longer used. HUVEC were grown endothelial cell growth medium (EGM (modified MCDB 131, 5% FBS, 0.04% hydrocortisone, 0.4% hFGF, 0.1% VEGF, 0.1% R3-IGF, 0.1% ascorbic acid, 0.1% hEGF, 0.1% GA-1000, 0.1% heparin, pH 7.35); BioWhitaker) to confluence in 12-well culture dishes in EGM medium. The HUVEC were then washed 3 times with HBSS. Lipid vesicles were made as in Example 1, but 1 mM carboxyfluorescein was loaded into the vesicles. The vesicles were then incubated with the cells for either 5, 10, 30, 45, 60, 90, 120 or 240 minutes at 37° C. in a humidified CO₂ incubator, after which the vesicles were washed from the cells, and the cells removed from the dish by gentle treatment with trypsin. The fluorescence of carboxyfluorescein in the HUVEC was measured using a Perkin-Elmer LS50B Luminescence Spectrophotometer (Wellesly, Mass.), using an excitation of 495 nm and emission of 520 nm. In some experiments, cells were not trypsinized, and photomicrographs of the cells were taken in order to demonstrate the homogeneity of the fusion event. The range of fluorescent units (FUs) for this experiment was 0 to 450 units. The rate of fusion highly depended on the lipid composition of the FUVs. DOPC showed little or no fusion at all for the first 30 minutes, after which the fusion rate became logarithmic, reaching approximately 350 FUs. In contrast, DOPC:DOPC-e (1:1) gave a much faster initial rate of fusion and a slower final rate of fusion (approximately 35 FUs at 5 minutes; approximately 100 FUs at 120 minutes). The fastest rate of fusion was found using DOPC:POPA (1:1), which showed significant delivery of ATP within 5 minutes. As designed, the fusion rate of the three vesicles can be characterized as fast, medium and slow.

To determine if vesicles were actually fusing with the cells or simply aggregating on the cell surface, HUVEC exposed to lipid vesicles and not removed from the culture wells were examined for the distribution of fluorescence by fluorescent microscopy. Cells exposed to all three compositions showed diffuse fluorescence throughout the cells after 60 minutes rather than punctate fluorescence, which would have suggested that lysosomes were sequestering the vesicles, thereby preventing cellular access to the carboxyfluorescein. Alternatively, the vesicles were aggregating on the cell surface. These results demonstrate that lipid vesicles fused to the cells and released the encapsulated contents within the cytoplasm rather than aggregating on the cell surface or being sequestered lysosomes.

To confirm the carboxyfluorescein studies, ³H-ATP was used as a tracer instead of the fluor. Vesicles were incubated with HUVEC for either 5, 10, 15, 30, 45, 60, 90, 120, or 240 minutes. DOPC gave the largest percent incorporation at this distant time period, followed by DOPC/DOPC-e, then ³H-ATP only, without vesicles. When the cells were washed repeatedly there was a significant change in the radioactivity of the cells. DOPC showed a slight but significant decrease in radioactivity; DOPC/DOPC-e showed no decrease in radioactivity after repeated washes, while free ³H-ATP showed a complete loss of radioactivity, confirming the observation that free ATP is unable to penetrate the cell membrane. These data, taken together with the fusion data, indicate that DOPC vesicles are being endocytosed, DOPC:DOPC-e vesicles are fusing, and free ATP does not enter cells. DOPC:POPA vesicles also could not be washed away, indicating that they also were fusing with cells and delivering the encapsulated contents into the cytoplasm.

### Example 3

### Endothelial Macromolecular Permeability

Any use of the vesicles of this invention to deliver encapsulated molecules in vivo or ex vivo into organs destined for transplantation requires that the vesicles and/or molecules must penetrate the vascular endothelium. The vascular endothelium constitutes a barrier, but the cell-to-cell barrier can be bridged, as for example, when leukocytes leave the circulation and enter the interstitial space. In order to address this issue, the effect of the lipid vesicles of this invention on endothelial permeability was measured.

HUVEC were grown to confluence on microporous filters (0.8 μm) in EGM. The cells were placed in a special chamber which allowed for the measurement of polypeptide flux across the endothelial monolayer. The tracer used to examine the effects of the lipid vesicles on endothelial permeability was FITC-albumin (1 mg/ml). The FITC-albumin and the lipid vesicles were added to the endothelial cells at time zero. Every 5 minutes, a 500 μl sample of the supernatant was collected and then analyzed for fluorescence using the Perkin-Elmer LS 50B Luminescence Spectrophotometer. DOPC vesicles had no effect on permeability, while HUVEC permeability increased in the presence of DOPC/DOPC-e, indicating that these vesicles created small gaps between adjacent endothelial cells.

### Example 4

### Fusion of Vesicles With Blood Vesicles

In an in vivo experiment, the ability of DOPC/POPA FUVs to fuse with endothelial cells of microvessels was examined. Using an isolated (single pedicle) rat cremaster muscle preparation and fluorescent intravital video microscopy, FUVs were observed to fuse to endothelial cells.

After the cremaster was isolated, blood was flushed with saline (Franken et al., 1996) and was followed by an infusion of FUVs made with fluorescent-labeled phospholipids (Molecular Probes). Immediately after infusion, the pedicle was clamped for 20 minutes, after which the microclamp was removed, and the cremaster allowed to reperfuse. As the free fluorescent FUVs were washed away by the blood flow, a visible fluorescent microvascular tree remained in which the edges of vessels showed a halo, indicating that fluorescent phospholipids had incorporated into the membranes of endothelial cells. After two hours of reperfusion, the intensity of the fluorescence remained unchanged suggesting that actual fusion of FUVs to the cell membrane had occurred rather than aggregating on the cell surface.

### Example 5

### Limb Reattachment

This example demonstrates the effectiveness in maintaining transplant viability when ATP is administered in FUVs.

Hind legs were amputated from rats, and the major feed arteries for the severed limbs were cannulated for infusion of A-FUV, loaded in a 1 mM ATP solution. The limbs were perfused with A-FUV or control solutions (see Table 1) every 3 hours, or as deemed necessary by the change in tissue ATP levels. The arterial pressure of the limbs were monitored during infusion to decrease the chance of flow-induced injury, and to monitor the overall preservation of the severed limbs (higher perfusion pressures may indicate limb morbidity). Following the preservation period, the limbs were flushed with Ringers to remove traces of A-FUV. The limbs were then surgically reattached, and external indices of limb function after anastomoses were evaluated (limb color, evidence of microthrombi, coagulation, limb temperature). The animals prior to and following replantation received heparin to prevent hemostasis. In addition, animals were placed on antibiotic therapy to reduce infection. Control limbs were perfused with vehicle only, vehicle and ATP only, or vehicle and FUVs only.

After 21 hours post-replantation, the A-FUV-treated limb exhibited a healthy pink color and had re-attained physiological temperature. After more than 150 days, those animals that received A-FUV-treated limbs were using these limbs as if the limb had never been amputated. The only qualitative side effect was a curling of the toes, most likely due to the lack of physical therapy, which most likely would have corrected this minor defect. In the controls, however, the limbs were darkly-colored and cold to the touch, exhibiting signs of necrosis. The summary of these results is shown in Table 1.

TABLE 1
Summary of results from limb replantation studies

| Group | Limb outcome | n |
|---|---|---|
| Vehicle only | necrosis | 2 |
| Vehicle and 1 mM ATP only | necrosis | 2 |
| Vehicle and FUVs only | necrosis | 2 |
| Vehicle and A-FUV | survival | 5 |

### Example 6

### A-FUVs Protect Isolated Hearts From Hypoxia

Like Example 5, this example demonstrates the protective effect of ATP-loaded FUVs on an organ destined for transplantation.

Hearts removed from rats were monitored using a Lagendorff heart perfusion apparatus. The hearts were cannulated and placed in a specially designed chamber which perfused the heart, and allowed for the injection of A-FUV. The oxygenated perfusate which was circulating to the heart was stopped, and A-FUV was injected into the heart. The heart was then placed in arrest by injecting a high potassium solution. The A-FUV was kept in the heart for 120 minutes at 37° C. under no-flow conditions. The heart was then flushed with oxygenated perfusate solution, and the performance of the heart was monitored. A-FUV treated hearts regained heart function compared to controls.

### Example 7

### Biotinylated Phospholipid Delivery

This example demonstrates, like Example 1, the generation of FUVs; but in this instance, biotinylated lipids are also incorporated.

Fusogenic vesicles that incorporate biotinylated phospholipids are introduced to the endothelial cell membranes. These biotin “tethers” are then used to functionalize the endothelial surface with an apoptosis-inducing ligand, such as FasL, thus creating a protective veil against activated T cells.

Fusogenic vesicles composed of DOPC/POPA and biotinylated lipids (N-biotinoyl-1,2-dihexadecanoyl-sn-glycero-3-phosphoethanolamine (BDGP; Molecular Probes; Eugene, Oreg.) at a ratio of 1:5, BDGP:DOPC phospholipids. HUVEC cultures (see Example 2; with the following modification: After 3-5 days, the cells were washed and placed in an experimental media (modified MCDB 131 (as available, for example, from Sigma; St. Louis, Mo.) with 2% heat-inactivated fetal bovine serum (FBS)) and incubated for 24 hours. The cells were washed 3 times with HBSS (pH 7.35). The effects of endotoxin contamination were mitigated by using disposable cellware, media and buffers low in endotoxin. The cells were then incubated with the fusogenic vesicles for 60 minutes (n=2) at 37° C., the culture washed three times with HBSS, and then 1 ml of fluorescent streptavidin (0.5 mg/ml; Molecular Probes) was added to the culture for 15 minutes, and the culture again washed three times. Fusogenic vesicles consisting of DOPC/POPA without incorporating biotinylated phospholipids served as a control (n=2). The result was a distinct difference in fluorescent staining between experimental and control cultures, demonstrating that the vesicles had fused to the HUVEC membranes.

### Example 8

### In Vivo Confirmation of Biotinylated FUV Fusion

The femoral vessels of a rat hind limb were dissected and prepared, followed by perfusion of the hind limb with 3 ml of fusogenic (DOPC/POPA) vesicles at 37° C., with (n=2), and without (controls n=2), biotinylated phospholipids. After 1 hour of incubation at 37° C., hind limbs were flushed with saline for 10 minutes, followed by perfusion with 1 ml of fluorescent streptavidin (0.5 mg/ml). Vessels were repaired and blood flow was reestablished. The animals for observed for 2 weeks, and then the endothelia were examined. As was the case for HUVEC cultures treated in the same way (see Example 7), striking difference between experimental animals and controls was observed, the experimental animal endothelial cells exhibiting bright fluorescence. These findings demonstrate that biotinylated phospholipids efficiently integrate into the membranes of endothelial cells when delivered as part of fusogenic vesicles.

### Example 9

### Optimizing Biotinylated Lipid Fusion Content of FUVs in Relationship to Fusogenecity (Prophetic Example)

This example provides a method for determining the optimal biotinylated lipid content in relationship to fusogenecity of the vesicles.

Results from other studies suggest that the higher the biotinylated lipid:non-biotinylated lipid ratio in FUVs, the less efficiently they fuse (Haque et al., 2001; Hu et al., 2001). The assay presented in this example allows for the determination of the maximal biotinylated phospholipid content that will not adversely affect delivery of biotinylated phospholipids to the membranes of endothelial cells. This assay determines those FUVs that incorporate the largest quantity of biotinylated phospholipids in cultured endothelial cells.

Fusogenic vesicles will be prepared that have various levels of biotinylation (Table 2). HUVEC cells will be prepared in culture dishes and incubated at 37° C. in 5% CO₂.

TABLE 2
FUV formulations*

| | BDGP/ | DOPC | POPA | BDGP |
|---|---|---|---|---|
| Formula | DOPC | (mg) | (mg) | (mg) |
| 1 | 1/1 | 2.5 | 0.10 | 3.24 |
| 2 | 1/5 | 2.5 | 0.06 | 0.648 |
| 3 | 1/25 | 2.5 | 0.052 | 0.1296 |
| 4 | 1/100 | 2.5 | 0.0505 | 0.0324 |
| 5 | 1/250 | 2.5 | 0.0502 | 0.01296 |
| 6 | 1/500 | 2.5 | 0.0501 | 0.00648 |

*Phospholipid molecular weights: DOPC (786.1), POPA (696.92), BDGP (1019.45)

Eight replications will be performed in 12-well plates coated with HUVECs. In each experiment, one well will be assigned to each of the six formulations. The remaining six wells will be used as controls. One ml of fusogenic vesicles will then be added to the HUVECs and then the cultures incubated for 1 hour. After washing the cells three times with Hank's Balanced Salt Solution (HBSS), the cultures will then incubated with 1 ml of fluorescent streptavidin at 0.1 mg/ml for 15 minutes. The cultures will then be washed three times. The cells will then be harvested using trypsin-EDTA for 0.5 minutes until the cells detach from the plate, at which point trypsin inhibitor will be added, and the cells counted in a hemacytometer. Finally, the amount of fluorescence per cell will be measured using a luminescence spectrophotometer. Because the number of FUVs being delivered to the cultures is the same in each case, and because the readings can be corrected for variations in cell number between samples and experiments, those samples that have the most fluorescence quantitatively represent optimum vesicle formulations.

### EXAMPLE 10

In vivo determination of optimized biotinylated lipid fusion content of FUVs in Relationship to Fusogenecity (Confirmation of In Vitro Results of Example 9) (Prophetic Example)

The formulation(s) found to be the most efficient at delivering the biotinylated phospholipids as will be determined in Example 9 will be prepared to test the life of the biotinylated lipids in vivo. Using a rat hind limb model, these experiments will determine whether biotinylated phospholipids that have been incorporated into the membranes of vascular endothelium can carry fluorescent streptavidin for a month.

The experimental design is summarized in Table 3.

TABLE 3
Experimental design

| | Experimental conditions | | | |
|---|---|---|---|---|
| | Perfusion | Vesicle | Follow-up | |
| Group | w/FUVs | biotinylation | (days) | Experimental role |
| 1a | No | No | 0 | Non-specific binding control for |
| 2a | Yes | No | 0 | streptavidin binding to endothelial cells |
| 3a | Yes | Yes | 0 | |
| 1b | No | No | 14 | Non-specific binding control for |
| 2b | Yes | No | 14 | streptavidin binding to endothelial cells |
| 3b | Yes | Yes | 14 | that have been exposed to FUVs |
| 1c | No | No | 28 | Experimental group to demonstrate |
| 2b | Yes | No | 28 | streptavidin binding to endothelial cells |
| 2c | Yes | Yes | 28 | exposed to biotinylated FUVs |

Preparation of Groups

General Procedure for all Groups

Wistar Furth (WF) rats will be anesthetized with sodium pentobarbital (60 mg/kg, administered intraperitoneally) and prepared for surgery by shaving the groin area. The femoral vessels will be dissected from the inguinal ligament to the birufcation of the inferior epigastric vessels. Murphy's branches will be ligated using 8-0 nylon. Heparin (0.5 cc; 1000 UI/ml) will be administered through the penile vein and allowed to circulated for 10 minutes. The hind limbs will then be separated from the body with exception of the bones and femoral and sciatic nerves. The arteries will be clamped and cannulated with a 24G catheter; and the veins clamped and severed distally from the clamps. The limb will then be flushed for 10 minutes with a heparinized Ringer's lactate solution (Ringer's; 1 Ul/ml).

Limbs will then be perfused with 3 ml of Ringer's, and the vessels clamped for an hour. The limbs will then be flushed for 10 minutes with Ringer's and then the limbs will then be perfused with 1 ml of streptavidin tagged with a fluorescent marker, such as fluorescein (0.1 mg; such as is available from Pierce; Rockford, Ill.). After 15 minutes of incubation, the limbs will again be flushed for 10 minutes with Ringer's.

Specific Treatments for Each Group

For Group 1a specimens, the limbs will be perfused with collagenase (2% dissolved in phosphate buffered saline), and the vessel will be clamped both distally and proximally. After 20 minutes, the distal clamp will be released, and the the endothelial cells will be flushed, which are then collected, to which 10% fetal bovine serum is added to inactivate collagenase. The endothelial cells will be washed (and re-suspended; streptavidin binding will be quantified by measuring fluorescence per cell.

The endothelial cells will be isolated from the hind limb circulation using collagenase. Briefly, the vessel will be briefly perfused with collagenase (2% dissolved in PBS), then clamped distally and proximally and incubated for a period of 20 minutes. The distal clamp will be released and the collagenase/cell suspension will be isolated. To inhibit the effects of the collagenase on the cells, a 10% FBS solution will be added to the isolate. The isolate will be washed (centrifugation at 200×g for 10 minutes, and then washed with PBS; repeated for a total of three washes). The cells will then be plated for analysis

For Groups 1b and 1c, the vessels will be re-anastomosed, the limb muscles and skin surgically reattached. The animals are observed for 14 (Group 1b) and 28 (Group 1c) days, after which the animals will be anesthetized, the vessels will be dissected and cannulated. As for Group 1a, endothelial cells are harvested with collagenase, and fluorescence measured.

For Groups 2a-2c, rats will be prepared in the same way as described for Groups 1a, b and c, except that the limbs will be perfused with 3 ml of fusogenic vesicles containing only DOPC/POPA (as determined in Example 9).

Because groups 1a-2c will be serving as negative controls, little fluorescence is expected to be observed; however, if significant fluorescence is observed, then non-specific binding of the streptavidin (or its fluorescent tag) is the likely culprit. In this case, pre-blocking non-specific binding sites may be incorporated into the procedure according to accepted protocols, and/or other fluorescent tags or sources of streptavidin will be assayed.

For Groups 3a-3c, rats will be prepared in the same way as described for Groups 1a, b and c, except that the limbs will be perfused with 3 ml of FUVs as determined in Example 9. Significant amounts of fluorescence are expected to be observed in all three groups, indicating that streptavidin has effectively bound to biotinylated lipids on endothelial cell surfaces and has been retained there for at least 4 weeks.

### Example 11

### Functionalization of Endothelial Cell Surfaces With a Lymphocyte-Apoptosis-Inducing Molecule (Exemplified by Recombinant(Chimeric) FasL) (Prophetic Example)

This and subsequent examples are designed to show the following:

(1) Endothelial cell membranes that have incorporated biotinylated lipids bind SA-FasL via the biotin tethers.

(2) The phospholipid-biotin-bound SA-FasL is functional.

To show that biotinylated phospholipids that are incorporated into endothelial membranes can bind SA-FasL chimeric polypeptide, endothelial cells are first contact with biotinylated FUVs and SA-FasL. Then, the number of cells in the exposed population are coated with SA-FasL will be determined. Finally, the mean number of attached SA-FasL molecules/cell will be ascertained. The experimental design is summarized in Table 4.

TABLE 4
Experimental design

| | Experimental | | | |
|---|---|---|---|---|
| | conditions | | | |
| | Vesicle | | | |
| Group | biotinylation | SA-FasL | n | Experimental role |
| 1 | No | Yes | 8 | Non-specific binding control |
| | | | | for SA-FasL binding to |
| | | | | endothelial cells. |
| 2 | No | Yes | 8 | Non-specific binding control |
| | | | | for SA-FasL binding to |
| | | | | endothelial cells that have |
| | | | | been exposed to FUVs |
| 3 | Yes | Yes | 8 | Experimental group to |
| | | | | demonstrate SA-FasL binding to |
| | | | | endothelial cells exposed |
| | | | | to biotinylated FUVs. |

Preparation of Groups

General Procedure for All Groups

HUVECs will be prepared and incubated for 1 hour with HBSS or biotinylated FUVs (formulation as determined in Example 9), as indicated in Table 4. Following incubation, the cells will be washed 3 times with HBSS and then incubated with 100 ng in 1 ml of SA-FasL (MW 32 kDa) for 15 minutes. Following incubation, the cells will be incubated with trypsin-EDTA for 0.5 minutes until the cells are released from the surface, at which point a trypsin inhibitor will be added. The cells will then be suspended, counted and concentrations normalized. After incubating with anti-FasL fluorescent pAb (Santa Cruz Biotech; Santa Cruz, Calif.) (Zhang et al., 1997) for 20 minutes, the cells will be washed 3 more times with HBSS.

To determine the efficiency of SA-FasL tethering, the cells will be subjected to fluorescence-activated cell sorting (FACS); to determine the efficiency of SA-FasL tethering per cell, the harvested cells are plated, and 50 cells are randomly selected and fluorescence quantified.

Because groups 1 and 2 will be serving as negative controls, little fluorescence is expected to be observed; however, if significant fluorescence is observed, then non-specific binding of the streptavidin (or its fluorescent tag) or of the anti-FasL pAb is the likely culprit. In this case, pre-blocking non-specific binding sites may be incorporated into the procedure according to accepted protocols, and/or other fluorescent tags or sources of streptavidin or anti-FasL antibodies will be assayed.

For group 3, 90-100% of cells are expected to be coated with SA-FasL at a density of >30,000 FasL molecules per cell.

### Example 12

### SA-FasL Induces Apoptosis in Activated T Cells When Tethered to Biotinylated Lipids

Activated T cells that contact SA-FasL-functionalized lymphocytes (splenocytes) (not endothelia) undergo apoptosis in a classic mixed lymphocyte reaction (MLR) assay (Shirwan et al., 1997; Yolcu et al., 2002). This example is designed to demonstrate that activated T cells that come in contact with SA-FasL tethered to phospholipids on endothelial cells, as opposed to lymphocytes, also undergo apoptosis.

These experiments exploit transplant immunorejection between two strains of rats, ACI and Wistar Furth. In three experimental groups, splenocytes from ACI-sensitized WF rats will be harvested and then exposed to endothelial ACI cells in vitro. Next, the WF splenocytes will be separated and adoptively transferred to a nude recipient rat (PVG rnu/rnu). The nude rat will then receive a transplant of an ACI-vascularized skin flap; rejection of the skin flap will then be monitored. The experimental design is summarized in Table 5.

TABLE 5
Experimental design

| | | Endothelial-Lymphocyte | | |
|---|---|---|---|---|
| | | Reaction | | |
| | | Stimulators | Responders | |
| Group | n | (endothelial) | (splenocytes) | Experimental role |
| 1 | 6 | ACI | ACI | Negative control, |
| | | | | demonstrating lack |
| | | | | of graft rejection |
| 2 | 6 | ACI | WF | Positive control, |
| | | | | demonstrating graft |
| | | | | rejection |
| 3 | 6 | ACI + SA- | WF | Experimental, demonstrating |
| | | FasL | | protective effect of SA-FasL, |
| | | | | resulting in diminished |
| | | | | graft rejection |

Group 1 (Rat Preparation and Surgical Procedures will be the Same for all Groups)

On Day −7, ACI rats will be injected with 10⁷ splenocytes harvested from 8-week old ACI rats. Free epigastric groin flaps from ACI rat donors will be raised as previously described (Fernandez-Botran et al., 2002). The flaps will measure 5 cm² and consist of skin, panniculus carnosus muscle, subcutaneous fat, epigastric fat pad, inguinal lymph nodes and femoral vessels. The animals will be anesthetized with sodium pentobarbital (60 mg/kg, administered intraperitoneally) and shaved in the groin area. Access to the flap will be by standard groin incision. The femoral artery and vein will be dissected from the inguinal ligament to the bifurcation of the inferior epigastric vessels. The distal ends of the femoral vessels will be ligated using 8-0 nylon. The isolated flap will then be flushed with heparinized lactated Ringer's solution through the femoral artery for 10 minutes; venous return will not be interrupted. At the end of flushing, the proximal ends of femoral vessels will be clamped, and the flap raised in its entirety with the epigastric vessels and fat pad.

On Day −4, nude rats will be transplanted with ACI skin flaps in the neck area. Nude rat recipients will be anesthetized with isoflourane 2-5% and shaved in the ventral aspect of the neck region. The right external carotid artery (ECA) and external jugular vein (EJV) will be exposed and the ECA carefully separated from the cervical sympathetic plexus, clamped proximally, and cut. The EJV will be clamped distally and cut. The flap will then be positioned in the neck area with four stay sutures, and vascular anastomoses will be performed between the femoral artery and the ECA and the femoral vein and the EJV. The skin will be closed using 6-0 nylon.

Also on Day −4, splenocytes will be harvested from the auto-sensitized ACI rats and exposed in a co-culture reaction to ACI endothelial cells. On day 0, the splenocytes will be isolated and assessed for apoptotic phenotypes using PI and annexin V-FITC (fluorescein) in Flow Cytometry. Ten million splenocytes will be injected into the penile vein of the nude rat recipient. The nude rat is observed for 28 days and biopsies of the skin flap taken on post-operative days 0, 2, 7 and 14, or when (if) signs of rejection or Graft-versus-Host Disease (GVHD) occur. On day 28, the rats will be euthanized, and samples of the skin flap, tongue, ear, liver and small bowel will be harvested. The latter will serve to assess the potential development of GVHD in the nude rat recipients.

In this group, there should be no rejection of the transplanted ACI skin flaps. However, if GVHD is observed, then PVG wt rats will be used (see below).

Group 2

On Day −7, WF rats will be injected with 10⁷ ACI splenocytes. On Day −4, nude rats will be transplanted with ACI skin flaps in the neck area. Also on Day −4, splenocytes will be isolated from the allosensitized WF rats and exposed in a co-culture to ACI endothelial cells. On day 0, the splenocytes will be harvested, and 10⁷ of them injected into the penile vein of the nude rat recipient. The rats are then observed as for Group 1. A strong rejection of the transplanted ACI skin flaps is expected.

Group 3

On Day −7, WF rats will be injected with 10⁷ ACI splenocytes. On Day −4, nude rats will be transplanted with ACI skin flaps in the neck area. Also on Day −4, splenocytes will be isolated from the allosensitized WF rats and exposed in a co-culture to ACI endothelial cells that will be coated with SA-FasL. On day 0, the splenocytes will be harvested, and 107 splenocytes injected into the penile vein of the nude rat recipient. The rats are then observed as for Group 1.

In all cases, if GVHD is observed, PVG wt rats instead of ACI rats as transfer donors in Group 1 and WF rats as transfer donors in Groups 2 and 3 will be used (Bell et al., 1990). Because PVG rats are syngeneic with the nude PVG recipients, any occurrence of GVHD should be avoided in all groups. In Group 1, absence of GVHD and rejection of the ACI skin flap would now be anticipated. In Group 2, rejection would be expected, and in Group 3, absence of rejection of the ACI skin flap would be expected.

### Example 13

### Demonstration That SA-FasL can Delay or Prevent Immune Rejection of a Transplanted Heart

This example demonstrates that SA-FasL, administered as per the invention, protects heart transplant recipients from immunorejection. The experimental design is summarized in Table 6.

TABLE 6
Experimental design

| | | Endothelial-Lymphocyte | | |
|---|---|---|---|---|
| | | Reaction | | |
| | | Stimulators | Responders | |
| Group | n | (endothelial) | (splenocytes) | Experimental role |
| 1 | 6 | ACI | WF | Negative control, |
| | | | | demonstrating |
| | | | | transplant rejection* |
| 2 | 6 | ACI + SA- | WF | Experimental, demonstrating |
| | | FasL | | protective effect of SA-FasL, |
| | | | | resulting in diminished |
| | | | | transplant rejection |

*Hearts are expected to be rejected around day 9, indicated by lack of beating (Askenasy et al., 2003).

Surgical Procedures

Donor Operation

Rats will be anesthetized (sodium pentobarbital 60 mg/kg, administered intraperitoneally), prepared and shaved, and 100 UI heparin will be injected into the infrahepatic inferior vena cava. The thorax will be opened through left and right dorsolateral incisions, and the diaphragm will be separated from the anterior chest wall. The right superior vena cava will then be ligated, and cut distally to the ligation. The first branch of the aorta will be ligated with 6-0 silk suture, and the suture will be retained to the first branch of the aorta. The left superior vena cava, pulmonary arteries, pulmonary veins, and azygos vein will then be ligated together with a 4-0 silk suture. Then, the heart will be harvested from the donor and stored in cold lactated Ringer's solution.

Recipient Operation

After shaving the neck of the recipient, a right anterolateral neck incision will be made. The skin will be separated from subcutaneous tissue in order to make a pocket for loosely accommodating the grafted heart. The jugular vein will then be isolated from its incoming branches, and the common carotid artery will be ligated from the subclavian artery as far as possible. Anastomoses between the right superior vena cava of the graft heart and the jugular vein of the recipient as well as between the donor aorta and the common carotid artery of the recipient will be performed using 10-0 nylon. After removing all the bulldog clamps and assuring that the beating graft is not congested, the incision will be closed with a one layer continuous suture.

Group 1

WF and ACI rats will be prepared and anesthetized on Day 0. Hearts will be harvested from the ACI donors, perfused with cold Ringer's lactate, clamped, incubated for 1 hour, and transplanted subcutaneously to the neck of WF recipients. The vessels that will be used for anastomosis will be the carotid artery and jugular vein. We will then follow the recipients for 12 weeks. Rejection will be monitored by palpating the neck and assessing whether the heart is still beating. At 12 weeks, or prior to that if the heart is rejected, the recipients will be euthanized, and blood and hearts will be harvested for histological studies to assess the grade of tissue rejection according to standard criteria and ascertain the presence of SA-FasL on the endothelium.

Group 2

Experiments will proceed as described for Group 1, except cold Ringer's lactate is replaced with a solution optimized fusogenic vesicles (as determined in Example 9) in cold Ringer's lactate. Following 1 hour of incubation, 100 ng of SA-FasL in 1 ml will be perfused in the coronary arteries, and then the transplantation completed.

### REFERENCES

Adler, S. H., and L. A. Turka. 2002. Immunotherapy as a means to induce transplantation tolerance. Curr.Opin.Immunol. 14:660-665.

Aral, H., S. Y. Chan, D. K. Bishop, and G. J. Nabel. 1997. Inhibition of the alloantibody response by CD95 ligand. Nat.Med. 3:843-848.

Askenasy, N., E. S. Yolcu, Z. Wang, and H. Shirwan. 2003. Display of Fas ligand protein on cardiac vasculature as a novel means of regulating allograft rejection. Circulation. 107:1525-1531.

Avila-Sakar, A. J., and W. Chiu. 1996. Visualization of beta-sheets and side-chain clusters in two-dimensional periodic arrays of streptavidin on phospholipid monolayers by electron crystallography. Biophys J. 70:57-68.

Bell, E. B., D. Rejali, E. H. Whitby, S. M. Sparshott, and C. P. Yang. 1990. Allograft rejection in athymic nude rats by transferred T cell subsets. II. The response of naive CD4+ and CD8+ thoracic duct lymphocytes to an isolated MHC class I disparity. Transplantation. 50:690-696.

Bellgrau, D., D. Gold, H. Selawry, J. Moore, A. Franzusoff, and R. C. Duke. 1995. A role for CD95 ligand in preventing graft rejection. Nature. 377:630-632.

Borisenko, G. G., T. Matsura, S. X. Liu, V. A. Tyurin, J. Jianfei, F. B. Serinkan, and V. E. Kagan. 2003. Macrophage recognition of externalized phosphatidylserine and phagocytosis of apoptotic Jurkat cells—existence of a threshold. Arch.Biochem.Biophys. 413:41-52.

Dubernard, J. M., E. Owen, G. Herzberg, M. Lanzetta, X. Martin, H. Kapila, M. Dawahra, and N. S. Hakim. 1999a. Human hand allograft: report on first 6 months. Lancet. 353:1315-1320.

Dubernard, J. M., E. Owen, G. Herzberg, X. Martin, V. Guigal, M. Dawahra, G. Pasticier, D. Mongin-Long, C. Kopp, A. Ostapetz, M. Lanzetta, H. Kapila, and N. Hakim. 1999b. [The first transplantation of a hand in humans. Early results]. Chirurgie. 124:358-365.

Fadok, V. A., A. de Cathelineau, D. L. Daleke, P. M. Henson, and D. L. Bratton. 2001. Loss of phospholipid asymmetry and surface exposure of phosphatidylserine is required for phagocytosis of apoptotic cells by macrophages and fibroblasts. J. Biol. Chem. 276:1071-1077.

Fernandez-Botran, R., V. Gorantla, X. Sun, X. Ren, G. Perez-Abadia, F. A. Crespo, R. Oliver, H. I. Orhun, E. E. Quan, C. Maldonado, M. Ray, and J. H. Barker. 2002. Targeting of glycosaminoglycan-cytokine interactions as a novel therapeutic approach in allotransplantation. Transplantation. 74:623-629.

Francois, C. G., W. C. Breidenbach, C. Maldonado, T. P. Kakoulidis, A. Hodges, J. M. Dubernard, E. Owen, G. X. Pei, X. P. Ren, and J. H. Barker. 2000. Hand transplantation: Comparisons and observations of the first four clinical cases. Microsurgery. 20:360-371.

Franken, R. J., F. W. Peter, G. L. Anderson, W. Z. Wang, P. M. Werker, D. A. Schuschke, M. Kon, and J. H. Barker. 1996. Anatomy of the feeding blood vessels of the cremaster muscle in the rat. Microsurgery. 17:402-408.

Green, D. R., and T. A. Ferguson. 2001. The role of Fas ligand in immune privilege. Nat.Rev.Mol.Cell Biol. 2:917-924.

Griffith, T. S., T. Brunner, S. M. Fletcher, D. R. Green, and T. A. Ferguson. 1995. Fas ligand-induced apoptosis as a mechanism of immune privilege. Science. 270:1189-1192.

Haque, M. E., T. J. McIntosh, and B. R. Lentz. 2001. Influence of lipid composition on physical properties and peg-mediated fusion of curved and uncurved model membrane vesicles: “nature's own” fusogenic lipid bilayer. Biochemistry. 40:4340-4348.

Helderman, J. H., and S. Goral. 2000. Transplantation for the millennium: attaining tolerance in our time—the Holy Grail. Semin.Nephrol. 20:83-86.

Hu, Q., C. R. Shew, M. B. Bally, and T. D. Madden. 2001. Programmable fusogenic vesicles for intracellular delivery of antisense oligodeoxynucleotides: enhanced cellular uptake and biological effects. Biochim.Biophys.Acta. 1514:1-13.

Ildstad, S. T., and D. H. Sachs. 1984. Reconstitution with syngeneic plus allogeneic or xenogeneic bone marrow leads to specific acceptance of allografts or xenografts. Nature. 307:168-170.

Janeway, C. A., P. Travers, M. Walport, and M. Shlomchik. 2001. Immunobiology. Garland Publishing, New York, N. Y.

Jones, J. W., S. A. Gruber, J. H. Barker, and W. C. Breidenbach. 2000. Successful hand transplantation. One-year follow-up. Louisville Hand Transplant Team. N.Engl.J.Med. 343:468-473.

Ju, S. T., D. J. Panka, H. Cui, R. Ettinger, M. el Khatib, D. H. Sherr, B. Z. Stanger, and A. Marshak-Rothstein. 1995. Fas(CD95)/FasL interactions required for programmed cell death after T-cell activation. Nature. 373:444-448.

Kabelitz, D., T. Pohl, and K. Pechhold. 1993. Activation-induced cell death (apoptosis) of mature peripheral T lymphocytes. Immunol. Today. 14:338-339.

Kang, S. M., D. B. Schneider, Z. Lin, D. Hanahan, D. A. Dichek, P. G. Stock, and S. Baekkeskov. 1997.

Fas ligand expression in islets of Langerhans does not confer immune privilege and instead targets them for rapid destruction. Nat.Med. 3:738-743.

Kann, B. R., D. W. Furnas, and C. W. Hewitt. 2000. Past, present, and future research in the field of composite tissue allotransplantation. Microsurgery. 20:393-399.

Kirk, A. D., L. C. Burkly, D. S. Batty, R. E. Baumgartner, J. D. Berning, K. Buchanan, J. H. Fechner, Jr., R. L. Germond, R. L. Kampen, N. B. Patterson, S. J. Swanson, D. K. Tadaki, C. N. TenHoor, L. White, S. J. Knechtle, and D. M. Harlan. 1999. Treatment with humanized monoclonal antibody against CD154 prevents acute renal allograft rejection in nonhuman primates. Nat.Med. 5:686-693.

Kirk, A. D., D. A. Hale, R. B. Mannon, D. E. Kleiner, S. C. Hoffmann, R. L. Kampen, L. K. Cendales, D. K. Tadaki, D. M. Harlan, and S. J. Swanson. 2003. Results from a human renal allograft tolerance trial evaluating the humanized CD52-specific monoclonal antibody alemtuzumab (CAMPATH-1H). Transplantation. 76:120-129.

Kirk, A. D., D. M. Harlan, N. N. Armstrong, T. A. Davis, Y. Dong, G. S. Gray, X. Hong, D. Thomas, J. H. Fechner, Jr., and S. J. Knechtle. 1997. CTLA4-1 g and anti-CD40 ligand prevent renal allograft rejection in primates. Proc.Natl.Acad.Sci. U.S.A. 94:8789-8794.

Kishimoto, K., V. M. Dong, and M. H. Sayegh. 2000. The role of costimulatory molecules as targets for new immunosuppressives in transplantation. Curr.Opin.Urol. 10:57-62.

Krammer, P. H., J. Dhein, H. Walczak, I. Behrmann, S. Mariani, B. Matiba, M. Fath, P. T. Daniel, E. Knipping, M. O. Westendorp, and. 1994. The role of APO-1-mediated apoptosis in the immune system. Immunol.Rev. 142:175-191.

Kuhl, T. L., J. Majewski, J. Y. Wong, S. Steinberg, D. E. Leckband, J. N. Israelachvili, and G. S. Smith. 1998. A neutron reflectivity study of polymer-modified phospholipid monolayers at the solid-solution interface: polyethylene glycol-lipids on silane-modified substrates. Biophys J. 75:2352-62.

Lau, H. T., M. Yu, A. Fontana, and C. J. Stoeckert, Jr. 1996. Prevention of islet allograft rejection with engineered myoblasts expressing FasL in mice. Science. 273:109-112.

Lenardo, M., K. M. Chan, F. Hornung, H. McFarland, R. Siegel, J. Wang, and L. Zheng. 1999. Mature T lymphocyte apoptosis—immune regulation in a dynamic and unpredictable antigenic environment. Annu.Rev.Immunol. 17:221-253.

Levine, B. L., W. B. Bernstein, N. E. Aronson, K. Schlienger, J. Cotte, S. Perfetto, M. J. Humphries, S. Ratto-Kim, D. L. Birx, C. Steffens, A. Landay, R. G. Carroll, and C. H. June. 2002. Adoptive transfer of costimulated CD4+ T cells induces expansion of peripheral T cells and decreased CCR5 expression in HIV infection. Nat Med. 8:47-53.

Li, X. K., T. Okuyama, A. Tamura, S. Enosawa, Y. Kaneda, S. Takahara, N. Funashima, M. Yamada, H. Amemiya, and S. Suzuki. 1998. Prolonged survival of rat liver allografts transfected with Fas ligand-expressing plasmid. Transplantation. 66:1416-1423.

Matsue, H., K. Matsue, M. Walters, K. Okumura, H. Yagita, and A. Takashima. 1999. Induction of antigen-specific immunosuppression by CD95L cDNA-transfected ‘killer’ dendritic cells. Nat.Med. 5:930-937.

Murray, J. E., J. P. Merrill, and J. H. Harrison. 1956. 6:432.

Nagata, S., and P. Golstein. 1995. The Fas death factor. Science. 267:1449-1456.

Pahler, A., W. A. Hendrickson, M. A. Kolks, C. E. Argarana, and C. R. Cantor. 1987. Characterization and crystallization of core streptavidin. J Biol Chem. 262:13933-7.

Rivnay, B., E. A. Bayer, and M. Wilchek. 1987. Use of avidin-biotin technology for liposome targeting. Methods Enzymol. 149:119-23.

Salama, A. D., G. Remuzzi, W. E. Harmon, and M. H. Sayegh. 2001. Challenges to achieving clinical transplantation tolerance. J.Clin.Invest. 108:943-948.

Sayegh, M. H. 1999. Finally, CTLA41g graduates to the clinic. J.Clin.Invest. 103:1223-1225. Sayegh, M. H., and L. A. Turka. 1998. The role of T-cell costimulatory activation pathways in transplant rejection. N.Engl.J.Med. 338:1813-1821.

Schneider, P., N. Holler, J. L. Bodmer, M. Hahne, K. Frei, A. Fontana, and J. Tschopp. 1998. Conversion of membrane-bound Fas(CD95) ligand to its soluble form is associated with downregulation of its proapoptotic activity and loss of liver toxicity. J.Exp.Med. 187:1205-1213.

Shirwan, H., L. Barwari, and D. V. Cramer. 1997. Rejection of cardiac allografts by T cells expressing a restricted repertoire of T-cell receptor V beta genes. Immunology. 90:572-578.

Suda, T., T. Takahashi, P. Golstein, and S. Nagata. 1993. Molecular cloning and expression of the Fas ligand, a novel member of the tumor necrosis factor family. Cell. 75:1169-1178.

Swenson, K. M., B. Ke, T. Wang, J. S. Markowitz, M. A. Maggard, G. S. Spear, D. K. Imagawa, J. A. Goss, R. W. Busuttil, and P. Seu. 1998. Fas ligand gene transfer to renal allografts in rats: effects on allograft survival. Transplantation. 65:155-160.

Sykes, M. 2001. Mixed chimerism and transplant tolerance. Immunity. 14:417-424.

Sykes, M., and D. H. Sachs. 1990. Bone marrow transplantation as a means of inducing tolerance. Semin.Immunol. 2:401-417.

Takeuchi, T., T. Ueki, H. Nishimatsu, T. Kajiwara, T. Ishida, K. Jishage, O. Ueda, H. Suzuki, B. Li, N. Moriyama, and T. Kitamura. 1999. Accelerated rejection of Fas ligand-expressing heart grafts. J.Immunol. 162:518-522.

Thomas, J. M., D. M. Neville, J. L. Contreras, D. E. Eckhoff, G. Meng, A. L. Lobashevsky, P. X. Wang, Z. Q. Huang, K. M. Verbanac, C. E. Haisch, and F. T. Thomas. 1997. Preclinical studies of allograft tolerance in rhesus monkeys: a novel anti-CD3-immunotoxin given peritransplant with donor bone marrow induces operational tolerance to kidney allografts. Transplantation. 64:124-135.

Thompson, L. D., and P. C. Weber. 1993. Construction and expression of a synthetic streptavidin-encoding gene in Escherichia coli. Gene. 136:243-6.

Tilney, N. 1996. Transplantation and Transplantation Biology: From legend to reality. In Transplantation Biology: Cellular and Molecular Aspects. N. Tilney, T. Srom, and L. Paul, editors. Lippincott-Raven, Philadelphia, Pa. 1-10.

Van Parijs, L., and A. K. Abbas. 1998. Homeostasis and self-tolerance in the immune system: turning lymphocytes off. Science. 280:243-248.

von Andrian, U. H. 2002. Immunology. T cell activation in six dimensions. Science. 296:1815-7.

Waldmann, H. 2002. A personal history of the CAMPATH-1H antibody. Med. Oncol. 19 Suppl:S3-S9.

Wekerle, T., J. Kurtz, H. Ito, J. V. Ronquillo, V. Dong, G. Zhao, J. Shaffer, M. H. Sayegh, and M. Sykes. 2000. Allogeneic bone marrow transplantation with co-stimulatory blockade induces macrochimerism and tolerance without cytoreductive host treatment. Nat.Med. 6:464-469.

Wekerle, T., M. H. Sayegh, J. Hill, Y. Zhao, A. Chandraker, K. G. Swenson, G. Zhao, and M. Sykes. 1998. Extrathymic T cell deletion and allogeneic stem cell engraftment induced with costimulatory blockade is followed by central T cell tolerance. J.Exp.Med. 187:2037-2044.

Yolcu, E. S., N. Askenasy, N. P. Singh, S. E. Cherradi, and H. Shirwan. 2002. Cell membrane modification for rapid display of proteins as a novel means of immunomodulation: FasL-decorated cells prevent islet graft rejection. Immunity. 17:795-808.

Zhang, H., Y. Yang, J. L. Horton, E. B. Samoilova, T. A. Judge, L. A. Turka, J. M. Wilson, and Y. Chen. 1997. Amelioration of collagen-induced arthritis by CD95 (Apo-1/Fas)-ligand gene transfer. J.Clin.Invest. 100:1951-1957.

Zhang, H. G., X. Su, D. Liu, W. Liu, P. Yang, Z. Wang, C. K. Edwards, H. Bluethmann, J. D. Mountz, and T. Zhou. 1999. Induction of specific T cell tolerance by Fas ligand-expressing antigen-presenting cells. J.Immunol. 162:1423-1430.

1. A method of treating a transplant, comprising:
administering to the transplant a vesicle, comprising:
(i) a T cell-apoptosis-inducing molecule, and
(ii) a phospholipid which is a stable vesicle former,
wherein the vesicle has a fusion rate of at least 20 vesicle fusions/second.

2. The method of claim 1, wherein the T cell-apoptosis-inducing molecule comprises a lipid moiety.

3. The method of claim 2, wherein the T cell-apoptosis-inducing molecule further comprises a biotin moiety.

4. The method of claim 3, wherein N-biotinoyl-1,2-dihexadecanoyl-sn-glycero-3-phosphoethanolamine comprises the lipid moiety.

5. The method of claim 3, wherein the T cell-apoptosis-inducing molecule comprises a chimeric polypeptide of avidin or streptavidin.

6. The method of claim 5, wherein the T cell-apoptosis-inducing molecule comprises a chimeric polypeptide of FasL.

7. The method of claim 1, wherein the transplant is heart or skin.

8. The method of claim 1, wherein the vesicle has a fusion rate of at least 10³ vesicle fusions/second.

9. The method of claim 1, wherein the vesicle further comprises ATP.

10. A method of treating a transplant, comprising:
administering to the transplant a vesicle, comprising:
(i) a phospholipid which is stable vesicle former,
(ii) at least one member selected from the group consisting of another polar lipid, PEG, a raft former and a fusion protein, and
(iii) a lipid,
wherein the vesicle has a fusion rate of at least 20 vesicle fusions/second.

11. The method of claim 10, wherein the T cell-apoptosis-inducing molecule comprises a lipid moiety.

12. The method of claim 11, wherein the T cell-apoptosis-inducing molecule further comprises a biotin moiety.

13. The method of claim 12, wherein N-biotinoyl-1,2-dihexadecanoyl-sn-glycero-3-phosphoethanolamine comprises the lipid moiety.

14. The method of claim 12, wherein the T cell-apoptosis-inducing molecule comprises a chimeric polypeptide of avidin or streptavidin.

15. The method of claim 14, wherein the T cell-apoptosis-inducing molecule comprises a chimeric polypeptide of FasL.

16. The method of claim 10, wherein the transplant is heart or skin.

17. The method of claim 10, wherein the vesicle has a fusion rate of at least 10³ vesicle fusions/second.

18. The method of claim 10, wherein the vesicle further comprises ATP.

19. A method treating a transplant, comprising administering to the transplant a T cell-apoptosis-inducing molecule.

20. A vesicle, comprising:
a phospholipid which is a stable vesicle former; and
a T cell-apoptosis-inducing molecule.

21. The vesicle of claim 20, wherein the T cell-apoptosis-inducing molecule comprises a lipid moiety.

22. The vesicle of claim 21, wherein the T cell-apoptosis-inducing molecule further comprises a biotin moiety.

23. The vesicle of claim 22, wherein N-biotinoyl-1,2-dihexadecanoyl-sn-glycero-3-phosphoethanolamine comprises the lipid moiety.

24. A vesicle, comprising:
(i) a T cell-apoptosis-inducing molecule,
(ii) a phospholipid which is stable vesicle former, selected from the group consisting of 1,2-dioleoyl-sn-glycero-3-phosphocholine, 1-palmitoyl-2-docosahexaenoyl-sn-glycero-3-phosphocholine and a mixture thereof, and
(iii) at least one member selected from the group consisting of a polar lipid which is not a stable vesicle former and PEG,
wherein the polar lipid which is not a stable vesicle former has a structure selected from the group consisting of formulas (XVII), (XVIII), (XIX), (XX), (XXI), (XXII), (XXIII), (XXV) and (XXVI) 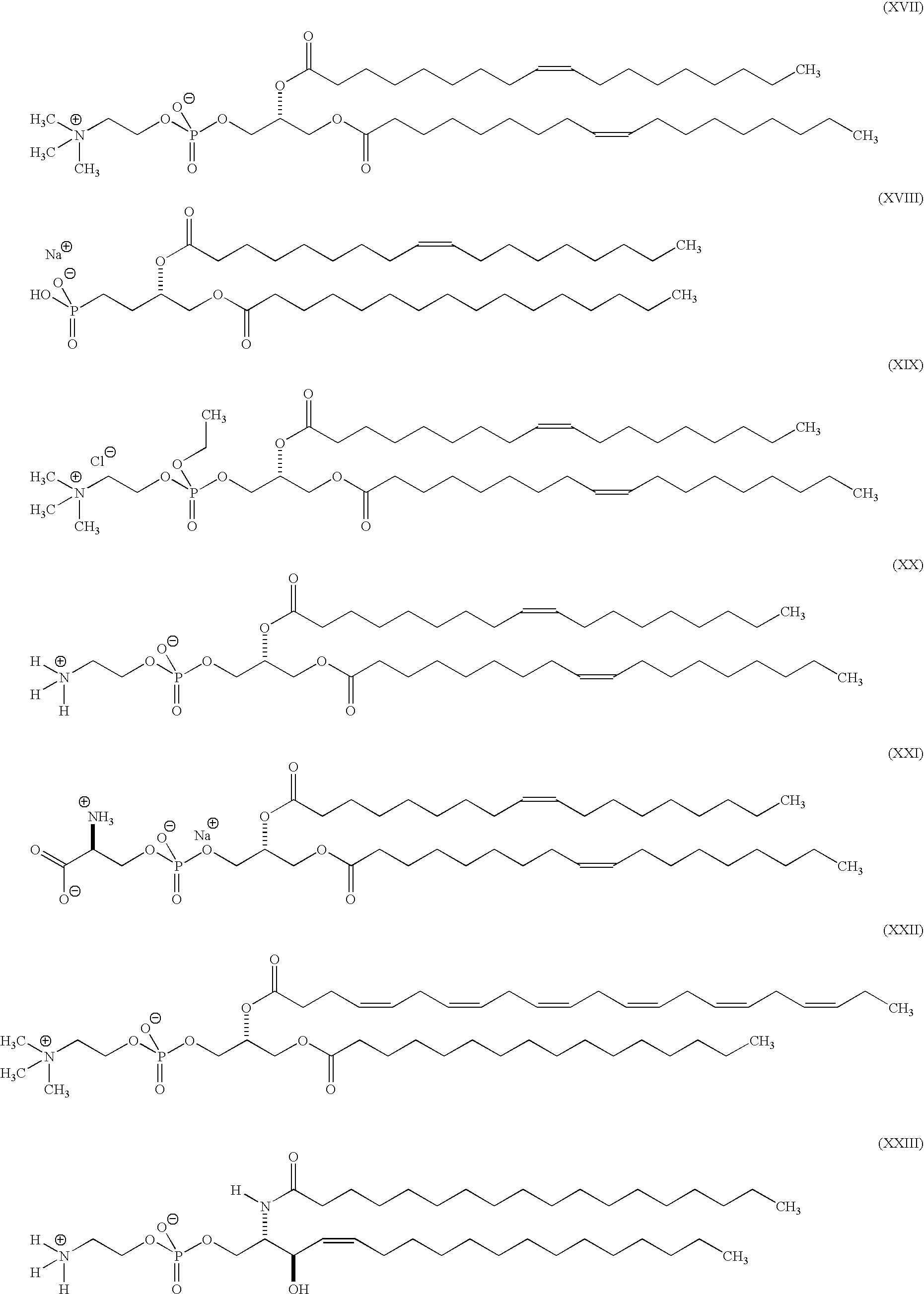
and
wherein the phospholipid which is stable vesicle former has a structure of formula (I)
X-L-Z₂ (I)
wherein X is H, or has a structure of formula (II) 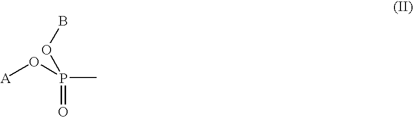
B is a cation or an alkyl group,
A is H, or has a structure selected from the group consisting of formulas (III), (IV), (V), (VI) and (VII) 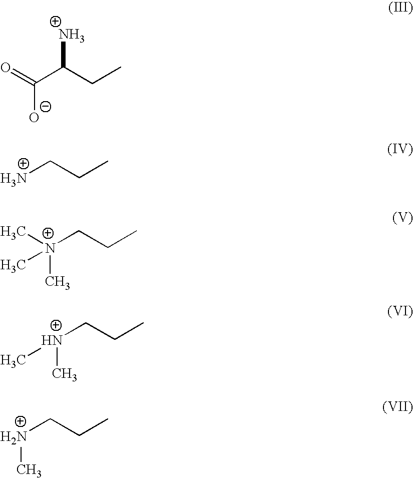
L has a structure selected from the group consisting of formulas (VIII), (IX) or (X) 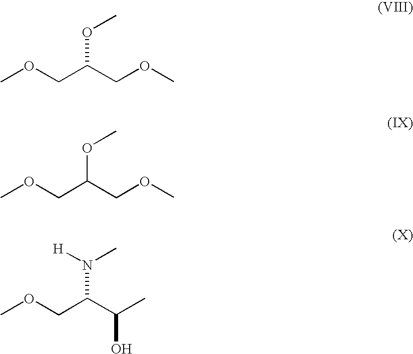
and E has a structure selected from the group consisting of (XII), (XIII), (XIV), (XV) or (XVI)

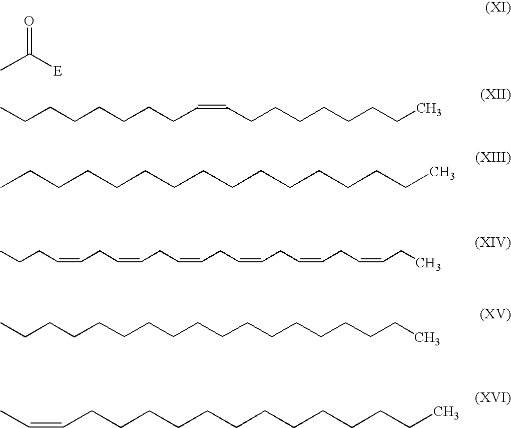

25. The vesicle of claim 22, wherein the T cell-apoptosis-inducing molecule comprises a chimeric polypeptide of avidin or streptavidin.

26. The vesicle of claim 25, wherein the T cell-apoptosis-inducing molecule comprises a chimeric polypeptide of FasL.

27. The vesicle of claim 20, wherein the transplant is heart or skin.

28. The vesicle of claim 20, wherein the vesicle has a fusion rate of at least 20 vesicle fusions/second.

29. The vesicle of claim 20, wherein the vesicle has a fusion rate of at least 10³ vesicle fusions/second.

30. The method of claim 20, wherein the vesicle further comprises ATP.

31. The vesicle of claim 20, wherein the lipid is N-biotinoyl-1,2-dihexadecanoyl-sn-glycero-3-phosphoethanolamine, and the T cell-apoptosis-inducing molecule is a chimeric polypeptide of a FasL polypeptide and at least one biotin-binding domain.

32. A transplant contacted with a vesicle of claim 20.

33. A method of transplanting a transplant, comprising:
contacting the transplant with a vesicle of claim 20; and
transplanting the transplant.

34. The method of claim 33, wherein the donor and recipient are immunohisto-incompatible.

35. A method of transplanting a transplant, comprising:
transplanting a transplant into a recipient without administering immunosuppressive therapy.

36. In a method of transplanting a transplant, including
transplanting a transplant into a recipient,
administering to the recipient immunosuppressive therapy, the improvement comprising:
contacting the transplant with a vesicle of claim 20.

37. A method of treating a transplant, comprising:
administering to the transplant:
a T cell-apoptosis-inducing molecule, and
a vesicle, comprising
(i) a means for binding the T cell-apoptosis-inducing molecule, and
(ii) a phospholipid which is a stable vesicle former,
wherein the vesicle has a fusion rate of at least 20 vesicle fusions/second.

38. The method of claim 37, comprising the sequential steps of:
(i) administering the vesicles to the transplant; and
(ii) administering the T cell-apoptosis-inducing molecule to the transplant.

39. A method of treating a transplant already transplanted according to the method of claim 37.


SEQUENCE LISTING (8 sequences)

SEQ ID NO: 1 - DNA, 399 bp, Artificial sequence
  artificial gene for streptavidin
gaattcatatggctgaagctggtatcaccggtacttggtacaaccagctggggtctaccttcatcgttaccgctggtgctgacggtgcactgaccggtacttacgaaagcgctgttggtaacgctgaaagccgttatgttctgaccggtcgttacgactctgctccggctaccgacggttctggtactgctctgggttggaccgttgcttggaaaaacaactaccgtaacgctcactctgctaccacctggtctggccagtacgttggtggtgctgaagctcgtatcaacacccagtggctgctgacctctggtaccaccgaagctaacgcttggaaatctaccctggttggtcacgacacgttcaccaaagttaaaccgtctgctgcttctatctaga

SEQ ID NO: 2 - protein, 128 aa, Artificial sequence
  polypeptide sequence of artificial gene for streptavidin (encoded by SEQ ID NO1)
MAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 3 - DNA, 604 bp, Gallus gallus
gaattccgcaaggagcacacccggctgtccacctgctgcagagatggtgcacgcaacctccccgctgctgctgctgctgctgctcagcctggctctggtggctcccggcctctctgccagaaagtgctcgctgactgggaaatggaccaacgatctgggctccaacatgaccatcggggctgtgaacagcagaggtgaattcacaggcacctacatcacagccgtaacagccacatcaaatgagatcaaagagtcaccactgcatgggacacaaaacaccatcaacaagaggacccagcccacctttggcttcaccgtcaattggaagttttcagagtccaccactgtcttcacgggccagtgcttcatagacaggaatgggaaggaggtcctgaagaccatgtggctgctgcggtcaagtgttaatgacattggtgatgactggaaagctaccagggtcggcatcaacatcttcactcgcctgcgcacacagaaggagtgaggatggccccgcaaagccagcaacaatgccggagtgctgacactgcttgtgatattcctcccaataaagctttgcctcagacaaaaaaaaaaaaaggaattc

SEQ ID NO: 4 - protein, 152 aa, Gallus gallus
MVHATSPLLLLLLLSLALVAPGLSARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 5 - DNA, 399 bp, Rattus norvegicus; identical to SEQ ID NO: 1

SEQ ID NO: 6 - protein, 278 aa, Rattus norvegicus
MQQPVNYPCPQIYWVDSSATSPWAPPGSVFSCPSSGPRGPGQRRPPPPPPPPSPLPPPSQPPPLPPLSPLKKKDNIELWLPVIFFMVLVALVGMGLGMYQLFHLQKELAELREFTNHSLRVSSFEKQIANPSTPSETKKPRSVAHLTGNPRSRSIPLEWEDTYGTALISGVKYKKGGLVINEAGLYFVYSKVYFRGQSCNSQPLSHKVYMRNFKYPGDLVLMEEKKLNYCTTGQIWAHSSYLGAVFNLTVADHLYVNISQLSLINFEESKTFFGLYKL

SEQ ID NO: 7 - DNA, 972 bp, Homo sapiens
tctagactcaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagatgttctaga

SEQ ID NO: 8 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL